(19) United States
(12) Patent Application Publication
(10) Pub. No.: US 2024/0059767 A1
(43) Pub. Date: Feb. 22, 2024

(54) TREATMENT OF DRY EYE SYNDROME USING AN OPHTHALMIC COMPOSITION COMPRISING TANFANERCEPT

(71) Applicant: HANALL BIOPHARMA CO., LTD., Daedeok-gu Daejeon (KR)

(72) Inventors: Seung Kook Park, Seoul (KR); Hyea Kyung Ahn, Yongin-si Gyeonggi-do (KR); Hye Eun Shim, Seoul (KR); Young Ju Kim, Seoul (KR); Sun Mi Park, Seoul (KR); Joo Hyun Chae, Suseong-gu Daegu (KR)

(21) Appl. No.: 18/261,368

(22) PCT Filed: Jan. 14, 2021
(86) PCT No.: PCT/KR2021/000564

§ 371 Date: Jul. 13, 2023

(51) Int. Cl. C07K 16/24; A61K 9/00; A61K 47/12; A61P 27/02

(52) U.S. Cl. CPC C07K 16/241; A61K 9/0048; A61K 47/12; A61P 27/02; A61K 2039/505

(57) ABSTRACT

The present invention relates to treatment of dry eye syndrome using a tanfanercept-containing ophthalmic composition. According to the present invention, as a result of clinical trial on dry eye syndrome, the tanfanercept-containing ophthalmic composition demonstrated excellent effects with regard to improvement of a central corneal staining score (CCSS) and a total corneal staining score (TCSS), which is clinically important. Further, such improvement of a central corneal staining score (CCSS) and a total corneal staining score (TCSS) was more significantly demonstrated in the patients with moderate-to-severe severity of dry eye. In addition, the patients with higher ocular discomfort score or higher eye dryness score, which are subjective symptoms, also showed greater improvement. Even though the same dose of tanfanercept was used, the tanfanercept-containing ophthalmic composition showed superior efficacy in the patient having more serious or severe baseline sign or symptom scores. This result was unexpected and is meaningful under the existing conditions without therapeutics for moderate or severe dry eye syndrome, since a novel and alternative therapeutic idea may be provided.

### FIELD OF INVENTION

The present invention relates to a treatment method of dry eye syndrome using an ophthalmic composition comprising tanfanercept.

### BACKGROUND OF INVENTION

Dry eye syndrome is a commonly encountered ocular disease affecting millions of people every year. In particular, it is known that postmenopausal women often suffer from dry eye syndrome due to hormonal changes at the end of the child-bearing period. Dry eye appears in different degrees depending on individuals. In the case of mild symptoms, a patient may feel discomfort, dryness and inconvenience. In severe cases, eyesight may be significantly damaged. Other diseases such as Sjogren's syndrome and cicatricial pemphigoid may exhibit combined symptoms of dry eye.

Based on results of the present studies, dry eye syndrome is understood as a disease caused by occurrence of autoimmune responses involving cytokines and antigen presenting cells on the ocular surface due to various stresses, which concentrates immune cells into corneal tissues, hence damaging the tissues.

Typical treatment methods of dry eye syndrome may include using artificial tear, thereby supplementing a tear film of an eyeball, reducing evaporation of tears and stabilizing the same. Further, an eye insert to stimulate interior tear production may also be used. Major components of artificial tears may include cellulose ether, carbomer, polyvinyl alcohol, polyvinyl pyrrolidone, sodium hyaluronate, etc., which are contained in a buffer or isotonic saline solution to prepare the artificial tear. These components may make a solution viscous in order to prevent the solution from easily flowing out of the eyes, otherwise, may prevent evaporation of tears while serving as a lubricant. However, a therapeutic agent including such components is not a fundamental solution for treatment but is limited to use as symptomatic treatment.

On the other hand, as the cause of dry eye is disclosed to be related to inflammatory response on the ocular surface, research into applying various types of anti-inflammatory substances to treatment is progressing and effects thereof have been proved.

Specifically, it has been reported that imbalance and excessive inflammatory mediators are expressed in ocular tissues, such as the lacrimal glands and the Meibomian glands, of patients suffering from dry eye syndrome. Further, a variety of compounds such as steroids, cytokine inhibitors, cyclosporine A, 15-HETE, etc. is known to be effective in treating dry eye syndrome.

Tumor necrosis factor alpha (TNFα) is a major factor involved in the inflammatory response and binds to human tumor necrosis factor receptor (TNFR) I or II on the cell surface to generate various cell reactions including apoptosis and inflammatory responses. Since it has been demonstrated that various autoimmune-related inflammatory responses can be treated by inhibiting the binding of TNFα and tumor necrosis factor receptor (TNFR), diverse TNFα inhibitors have been developed and representative TNFα inhibitors include, for example, etanercept (trade name, Enbrel) produced by binding Fc to soluble TNFR II, infliximab (trade name, Remicade) and adalimumab (trade name, Humira) which are antibodies against TNFα, or the like. These are mostly used for treatment of rheumatoid arthritis, psoriasis and Crohn's disease.

However, the anti-TNFα antibody drug entails a limitation in that it cannot efficiently reach an inflammatory disease occurred on local sites due to high molecular weight. Accordingly, the present applicant has developed a polypeptide molecule having a small size but high activity, which is suitable for treatment of local inflammatory disease. The TNFα inhibitor of the present invention, that is, modified human tumor necrosis receptor-1 polypeptide, tanfanercept, was disclosed in Korean Patent Laid-Open Publication No. 2012-0072323 filed by the present applicant. Further, a use of tanfanercept for treatment of dry eye syndrome has also been described in Korean Patent Laid-Open Publication No. 203-0143484.

### PRIOR ART LITERATURE

### Patent Document

Korean Patent Laid-Open Publication No. 2012-0072323
Korean Patent Laid-Open Publication No. 2013-0143484

### SUMMARY OF INVENTION

### Technical Problem to be Solved

An objective of the present invention is to propose a patient group suitable for treatment of dry eye syndrome using a tanfanercept-containing ophthalmic composition.

Another objective of the present invention is to provide an ophthalmic composition with appropriate constitutional composition for treatment of dry eye syndrome, which uses tanfanercept, and a method for treatment of dry eye syndrome using the same ophthalmic composition.

### Technical Solution

Tanfanercept is a polypeptide consisting of total 171 amino acids. Therefore, similar to general protein-containing pharmaceutical compositions, one of the most important issues in drug development is to ensure stability before administration to a patient in order to achieve maximum efficacy of the composition. Another important issue in drug development is to find a specific patient group with best response to the drug in addition to ensuring drug stability, so as to improve utility of the drug. Even if the drug has been recognized to have validity, the efficacy may not apply to all patients. Therefore, in order to provide information on which a most suitable drug for treatment of a disease of a patient is selectable, it is preferable to designate an appropriate patient group that may be treated with the corresponding drug.

Since the use of tanfanercept for the treatment of dry eye syndrome has been found, the present inventors have continuously studied to manufacture an ophthalmic composition for treatment of dry eye syndrome using the above drug. In view of medicinal properties of tanfanercept as a protein medicament, constitutional composition of the ophthalmic composition to improve drug stability was subjected to research, and the patient group showing the highest treatment efficiency when treated using tanfanercept was determined through clinical trial.

As a result, the present invention provides an ophthalmic composition comprising tanfanercept as an active ingredient for treatment of dry eye syndrome in patients having moderate to severe dry eye.

According to results of the clinical trial for tanfanercept, it was confirmed that an ophthalmic composition comprising tanfanercept exhibits superior effects on treatment of dry eye syndrome in patients having moderate to severe dry eye rather than patients having mild dry eye. This result is contrary to prior art in which most ophthalmic compositions for treatment of dry eye syndrome have been assessed to be suitable for treating mild dry eye rather than moderate to severe dry eye. The fact that the ophthalmic composition comprising tanfanercept shows more excellent effects on treatment of dry eye syndrome in patients having moderate to severe dry eye rather than patients having mild dry eye will substantially provide a novel treatment means in the field of ophthalmic compositions for treatment of dry eye syndrome.

Patients with moderate-to-severe dry eye may include patients who have a corneal staining score of 2 or more on at least one region of Inferior Cornea, Central Cornea and Superior Cornea, and a Schirmer's tear test score of not less than 1 and not more than 7.

According to the present invention, a sign as an objective assessment indicator of dry eye syndrome and a symptom as a subjective assessment indicator of the same may be used as a method of evaluating a degree of dry eye in a patient having dry eye and a degree of improvement of the dry eye by a medicine.

The objective assessment indicator, that is, the sign, may be assessed by evaluation of a corneal staining score (“CSS”). CSS evaluation is a method of dropping a staining agent in an eye and then observing a degree of damage to the cornea, followed by scoring the observed damage degree. CSS is represented by a score ranging from 0 to 4. The degree of staining is proportional to the degree of damage. That is, a higher CSS indicates more serious damage of the cornea. CSS of 2 indicates mild in terms of standard for evaluation of CSS. Likewise, CSS of 3 indicates moderate while CSS of 4 defines severe. However, even when CSS of any one region is 2, the dry eye syndrome may be in a moderate level or more in terms of other objective signs or subjective assessment indicator symptoms. Therefore, a state of the dry eye should be determined in combination with other evaluation indicators in addition to CSS.

Another objective assessment indicator determined along with CSS may be the Schirmer's tear test score. The Schirmer's tear test (“STT”) is a method in which an STT strip is placed on the brim of the lower eyelid of each eye of a patient, followed by closing the eye for 5 minutes, and then, the STT strip is removed, and a length (mm) of the wet portion is measured and recorded. In the present invention, the patient having moderate to severe dry eye may include patients having CSS of 2 or more on at least one region of Inferior Cornea, Central Cornea and Superior Cornea and an STT score in the range of 1 to 7. Herein, it means that the lower STT score, the more severe the severity of the dry eye.

When the ophthalmic composition of the present invention is administered to the patient having dry eye syndrome, improvement in signs of moderate to severe dry eye may begin to appear within 8 weeks after administration of the ophthalmic composition.

The period of 8 weeks after administration is only illustrative and improvement in signs (“sign improvement”) may be expressed, for example, within a shorter period of time such as 7 weeks, 6 weeks, 5 weeks, 4 weeks, 3 weeks, 2 weeks or 1 week after administration. This means that sign improvement depends on individuals, that is, patients, and may vary on the basis of different factors such as severity, dose of drug, and etc., provided that the sign improvement appears at least within 8 weeks after administration.

Specifically, sign improvement may be determined by reduction in a total corneal staining score (TCSS) and a central corneal staining score (CCSS). The corneal region having high CSS may be different depending upon patients or severity of dry eye syndrome. However, the total corneal staining score (TCSS) includes the sum of CSSs on each regions of the cornea, including inferior, central, and superior regions, so that reduction in TCSS is significantly meaningful in regard to sign improvement. Generally, when an ophthalmic composition is administered in the form of an eye drop, the eye drop may settle to the inferior corneal portion, thus increasing the possibility of improvement in an inferior corneal staining score (ICSS). However, the present invention will adopt reduction in a central corneal staining score (CCSS), a clinically relevant region directly related to vision, as an indicator for sign improvement.

Meanwhile, whether the moderate to severe dry eye is improved or not may also be assessed by improvement of symptoms as the subjective assessment indicator.

When the ophthalmic composition of the present invention is administered to the patient having dry eye syndrome, improvement in symptoms of the moderate to severe dry eye may begin to appear within 8 weeks after administration of the ophthalmic composition.

The period of 8 weeks after administration is only illustrative and improvement in symptoms (“symptom improvement”) may be expressed, for example, within a shorter period of time such as 7 weeks, 6 weeks, 5 weeks, 4 weeks, 3 weeks, 2 weeks or 1 week after administration. This means that symptom improvement depends on individuals, that is, patients, and may vary on the basis of different factors such as severity, dose of drug, etc., provided that the symptom improvement appears at least within 8 weeks after administration.

The symptom improvement may be determined by reduction in eye dryness score (EDS). The EDS used in the present invention is defined by subjects' evaluation of eye dryness score on the visual analog scales (VAS). EDS of subjects to be tested may be evaluated on a continuous scale in the range of 0 to 100, wherein 0 refers to “no dryness” while 100 indicates “maximum dryness”.

According to an embodiment of the present invention, the patient may have CCSS of 2 or more (CCSS>2). CCSS>2 means that CSS at the central corneal region is a mild level or more. Further, the patient may have TCSS of 5 or more (TCSS>5). TCSS>5 means that the sum of CSSs on the inferior, central and superior cornea regions is a mild level or more.

Further, according to an embodiment of the present invention, the patient may have a Schirmer's tear test score (“STTS”) in the range of 1 to 7 before administration of the ophthalmic composition. According to another embodiment of the present invention, the patient may have STTS in the range of 1 to 3 before administration of the ophthalmic composition. As described above, the STTS is inversely proportional to the severity of dry eye syndrome. The ophthalmic composition according to the present invention also exhibits excellent dry eye treatment effects in the case of patients having STTS in the range of 1 to 3.

According to another embodiment, the patient may have an ocular discomfort score (“ODS”) of 3 or more before administration of the ophthalmic composition. The ODS is determined by scoring a degree of ocular discomfort subjectively sensed by the patient in the range of 0 to 4 points. Therefore, a higher score means increased ocular discomfort. The ODS of 3 or more indicates that the patient has at least moderate dry eye syndrome and hence feels intermittent and even continuous discomfort.

According to another embodiment of the patient group suitable to administration of the ophthalmic composition of the present invention, the patient may have TCSS of 5 or more and ODS of 3 or more before administration of the ophthalmic composition.

Alternatively, the patient suitable to administration of the ophthalmic composition of the present invention may include patients having TCSS of 4 or more and EDS of 40 or more, for example, TCSS of 5 or more and EDS of 60 or more before administration of the ophthalmic composition.

Further, the patient suited to administration of the ophthalmic composition of the present invention may include patients having experience of using artificial tears within 1 month before administration of the ophthalmic composition.

The ophthalmic composition of the present invention may be formulated in the form of an eye drop.

Specifically, the ophthalmic composition of the present invention may be administered once or more per day in the form of an eye drop. For example, the ophthalmic composition may be administered twice per day in the form of an eye drop.

Meanwhile, in order to develop an ophthalmic composition that can minimize production of tanfanercept-derived impurities (acidic/basic variants) even if the composition is stored under cold storage conditions and/or even under accelerated and stressed conditions, many studies on stability of tanfanercept have been conducted by the present inventors. In particular, the present inventors have investigated diverse formulations including buffers, isotonic agents, pH range, functional excipients, or the like. As a result, it was found that using a stabilizer such as histidine or sucrose induces production of acidic/basic variants at specific pH, thereby exerting effects on stability of the tanfanercept composition. Therefore, the present invention intends to provide a tanfanercept containing ophthalmic composition that is stable at pH 5.0 to pH 6.5 without including a stabilizer.

Specifically, the present invention provides a stable ophthalmic composition containing tanfanercept, which comprises tanfanercept and a buffer system at pH 5.0 to pH 6.5 and substantially does not contain any stabilizer.

Tanfanercept is a TNFRI variant disclosed in Korean Patent Laid-Open Publication No. 2013-0143484, and is represented by an amino acid sequence including a modified amino acid L68V/S92M/H95F/R97P/H98G/K161N among the amino acid sequence TNFRI171 consisting of amino acid sequences No. 41 to No. 211 of natural TNFRI.

Tanfanercept is a polypeptide consisting of a total of 171 amino acids. Therefore, similar to a general protein-containing pharmaceutical composition, one of the most important issues in development of drug is to ensure stability before administration to patients in order to express best efficacy of the pharmaceutical composition.

In order to develop a formulation ensuring stability of tanfanercept, first of all, the present inventors have conducted experiments for storage stability of tanfanercept. As a result of the experiments, it was observed that tanfanercept forms a charge variant during storage (Experimental Example 1). The term “charge variant” mentioned herein refers to a change in charge of a protein or polypeptide due to modification of the protein or polypeptide from a natural state thereof. In some examples, the charge variant may have higher acidity, that is, lower pI value than that of the original protein or polypeptide. In other examples, the charge variant may have higher basicity, that is, higher pI value than that of the original protein or polypeptide. The above modification may be a result of manipulation or natural processes, for example, oxidation, de-amidation, processing of C-terminal in lysine residue, formation of N-terminal pyroglutamate and non-enzymatic saccharification. In some examples, the charge variant of protein or polypeptide may be a glucoprotein, in which glycan adhered to a protein is modified by addition of sialic acid or a derivative thereof to change a charge of the glucoprotein, compared to parent glucoprotein. The term “tanfanercept charge variant” mentioned herein refers to a substance in which a charge of tanfanercept is changed due to modification of tanfanercept from a natural state thereof.

It is well known that the charge variant generally causes deterioration in activity of a drug. Therefore, it is necessary to minimize the production amount of the charge variant to a predetermined level or less. Therefore, the present inventors have investigated whether generation of impurities such as the charge variant can be minimized using come components useable as ophthalmic stabilizers and, as a result, confirmed that it is preferable to include sucrose and histidine at first (Experimental Example 2). However, in further research to discover proper pH of the tanfanercept-containing composition, it was found that the production rate of a charge variant is high at specific pH even when these stabilizers are used. On the contrary, it has been demonstrated that adjusting pH in the range of pH 5.0 to pH 6.5 without using any stabilizer is a desirable solution to decrease the production rate of the charge variant as much as possible (Experimental Example 3).

Accordingly, the present invention may provide an ophthalmic composition containing tanfanercept, which comprises tanfanercept and a buffer system at pH 5.0 to pH 6.5 and substantially does not include a stabilizer.

Tanfanercept is preferably included with a proper content in the composition. As the content increases, a content of impurities such as aggregates may be increased. With regard to the tanfanercept-containing ophthalmic composition, tanfanercept may be included in an amount of 0.01% (weight by volume, w/v) to 1% (w/v), for example, 0.02% (w/v) to 1% (w/v), 0.05% (w/v) to 0.8% (w/v), 0.1% (w/v) to 0.7% (w/v), 0.2% (w/v) to 0.6% (w/v), etc. In consideration of commercial purposes, tanfanercept may be included in an amount-of 0.25% (w/v), 0.5% (w/v), 1% (w/v), etc. in the composition. The content of tanfanercept may depend on types of diseases of patients as a subject for drug administration, severity of the disease, etc.

The tanfanercept-containing ophthalmic composition according to the present invention may comprise a buffer system at pH 5.0 to pH 6.5. It is sufficient that the buffer system has pH in the range of pH 5.0 to pH 6.5. The numerical values within the range of pH 5.0 to pH 6.5, for example, the buffer system at pH 5.0 to pH 6.0, the buffer system at pH 5.5 to pH 6.5, the buffer system at pH 5.5 to pH 6.0, the buffer system at pH 5.8 to pH 6.3, or the like, may be included within the scope of the present invention. According to one embodiment of the present invention, the tanfanercept-containing ophthalmic composition of the present invention may include a buffer system at pH 5.0 to pH 6.0. According to another embodiment of the present invention, the tanfanercept-containing ophthalmic composition of the present invention may include a buffer system at pH 5.5 to pH 6.0.

A method of implementing the buffer system in the tanfanercept-containing ophthalmic composition of the present invention is well known to those skilled in the art. The buffer system at pH 5.0 to pH 6.5 may include, for example, one or more buffers selected from a group consisting of a phosphate buffer, a histidine buffer, an acetate buffer, a succinate buffer, a citrate buffer, a glutamate buffer and a lactate buffer. It is considered that stability of tanfanercept may be ensured using any buffer system if the condition of pH 5.0 to pH 6.5 is satisfied. In fact, specific buffer systems are preferably used, as compared to others. From the following embodiments, it could be confirmed that, as compared to the acetate buffer, the citrate buffer is relatively advantageous in the aspect of controlling production of aggregates or charge variants (Experimental Example 4). Therefore, according to embodiments of the present invention, the buffer system may be a buffer system including a citrate buffer, for example, a citrate buffer system or a citrate-phosphate buffer system, without being limited thereto. The buffer included in the buffer system may be composed of a combination of conjugate acid-conjugate base in order to increase buffer effects. For instance, according to an embodiment of the present invention, the buffer system may include a citrate buffer wherein the citrate buffer includes trisodium citrate (conjugate base) and citric acid (conjugate acid).

The buffer system of the present invention may include a buffer in a concentration of 5 mM to 50 mM, for example, a buffer in a concentration of 10 mM to 30 mM.

The ophthalmic composition of the present invention is characterized by substantially not including a stabilizer. Herein, the stabilizer refers to an additional component included in a formulation in order to prevent reduction of chemical or physical stability or biological activity of tanfanercept which is used as an active ingredient. For example, with regard to the ophthalmic composition, it is well known that saccharides such as sucrose or mannitol may be used to inhibit coagulation of protein, or an amino acid-containing stabilizer such as proline, arginine, glycine, lysine or methionine may be used to attain stabilization of protein. As demonstrated in the following embodiments, the present invention is different from typical cases and it was found that, when the stabilizer as described above is included, it adversely affects stability of tanfanercept. Therefore, the ophthalmic composition of the present invention is substantially free of a stabilizer. The buffering agent or isotonic agent mentioned in the present invention does not qualify as a stabilizer.

The expression of “substantially free” of the stabilizer means that the stabilizer is included in less than 0.1% (w/v), less than 0.05% (w/v), less than 0.03% (w/v), less than 0.02% (w/v), less than 0.01% (w/v), less than 0.005% (w/v), or less than 0.001% (w/v). Most preferably, it means that it does not contain any stabilizers.

An osmotic concentration of the ophthalmic composition may range from 260 mOsm/kg to 320 mOsm/kg.

The ophthalmic composition of the present invention may further comprise an isotonic agent in addition to tanfanercept as an active ingredient and a buffer system. The isotonic agent may be used to adjust an osmotic pressure of the ophthalmic composition according to the present invention. According to the present invention, the isotonic agent may be included to allow the ophthalmic composition of the present invention to have an osmotic concentration of 260 mOsm/kg to 320 mOsm/kg. The osmotic pressure is determined by measuring the number of particles dissolved per unit of water. In the solution, as the number of solute particles is decreased in proportion to unit number of water (solvent), a low osmotic pressure solution is less concentrated. When solutions having different solute concentrations are separated using a semi-permeable membrane (a membrane through which only solvent molecules can pass), osmosis may occur such that the solvent molecules pass through the membrane and intersect between a low concentration and a high concentration, thus resulting in balance of concentration. A pressure of driving the above movement is called osmotic pressure, which is controlled by the number of “particles” of the solute. Further, a solution containing particles at the same concentration and applying the same osmotic pressure is determined to have iso-osmotic pressure. If low-osmotic or high-osmotic pressure solution enters an eye, the eye may be in danger of damage. Therefore, an iso-osmotic pressure solution used for an ophthalmic medicine is needed. In the present invention, sodium chloride is used as an isotonic agent. With regard to the ophthalmic composition of the present invention, a content of the isotonic agent may range from 0.5% (w/v) to 1% (w/v). Further, a concentration of the isotonic agent used for the ophthalmic composition of the present invention may range from 100 mM to 150 mM.

According to one embodiment of the present invention, the ophthalmic composition of the present invention may include tanfanercept, a buffer system at pH 5.5 to pH 6.0, an isotonic agent and water. In one example, the ophthalmic composition of the present invention may include tanfanercept, a buffer system containing a citrate buffer at pH 5.5 to pH 6.0, sodium chloride and water.

The tanfanercept-containing ophthalmic composition according to the present invention may be very stable under accelerated or stress conditions.

The tanfanercept-containing ophthalmic composition according to the present invention may contain charge variants in an amount of 20% or less after storage for 6 months under accelerated conditions.

The tanfanercept-containing ophthalmic composition according to the present invention may contain basic variants in an amount of 10% or less after storage for 6 months under accelerated conditions.

The tanfanercept-containing ophthalmic composition according to the present invention may contain acidic variants in an amount of 10% or less after storage for 6 months under accelerated conditions.

The tanfanercept-containing ophthalmic composition according to the present invention may contain charge variants in an amount of 20% or less after storage for 36 months under long-term storage conditions.

The tanfanercept-containing ophthalmic composition according to the present invention may contain basic variants in an amount of 10% or less after storage for 36 months under long-term storage conditions.

The tanfanercept-containing ophthalmic composition according to the present invention may contain acidic variants in an amount of 10% or less after storage for 36 months under long-term storage conditions.

Further, the present invention may provide a method for treatment of dry eye syndrome, which includes administering an ophthalmic composition comprising tanfanercept as an active ingredient to a patient with moderate-to-severe dry eye.

The ophthalmic composition comprising tanfanercept as an active ingredient is substantially the same as described in the above embodiments, and the patient suited to administration of the ophthalmic composition is also the same as described above.

A pharmaceutical composition for ophthalmic use according to the present invention may improve physicochemical stability and biological stability of tanfanercept, and can be administered to patients suffering from TNF-mediated eye diseases such as dry eye syndrome by any typical administration method such as ophthalmic instillation.

### Effect of Invention

According to the present invention, as a result of clinical trial on dry eye syndrome, the tanfanercept-containing ophthalmic composition demonstrated excellent effects with regard to improvement in a central corneal staining score (CCSS) and a total corneal staining score (TCSS), which are clinically important. Further, such improvement of a central corneal staining score (CCSS) and a total corneal staining score (TCSS) was more significantly demonstrated in patients having moderate to severe dry eye. In addition, patients having higher ocular discomfort score and higher eye dryness score, which are subjective symptoms, also showed greater improvement. Even if the same dose of tanfanercept was used, the tanfanercept-containing ophthalmic composition showed superior efficacy in the patient having more serious or severe sign or symptom. This result was unexpected and is meaningful under the existing conditions without therapeutics for moderate or severe dry eye syndrome, since a novel and alternative therapeutic idea may be provided.

### BRIEF DESCRIPTION OF DRAWINGS

FIG. 1 shows results of isoelectric focusing (IEF) of tanfanercept after storage for 0 to 4 weeks at 37° C.

FIG. 2 illustrates results of IEX-HPLC analysis of tanfanercept after storage for 0 to 4 weeks at 37° C.

FIG. 3 shows results of IEF analysis of charge variants.

FIG. 4 illustrates results of IEX-HPLC analysis of charge variants.

FIG. 5 illustrates changes in IEX-HPLC main peaks of the control group without any stabilizer and the stabilizer screening solution groups which contain methionine, glycine, histidine hydrochloride and sucrose, respectively, as a stabilizer.

FIG. 6 illustrates changes in IEX-HPLC acidic variants of the control group without any stabilizer and the stabilizer screening solution groups which contain methionine, glycine, histidine hydrochloride and sucrose, respectively, as a stabilizer.

FIG. 7 illustrates changes in IEX-HPLC basic variants of the control group without any stabilizer and the stabilizer screening solution groups which contain methionine, glycine, histidine hydrochloride and sucrose, respectively, as a stabilizer.

FIG. 8 illustrates results of analyzing stability of the tanfanercept-containing ophthalmic composition after 4 weeks of a storage at 40° C., in terms of change in RP-HPLC variant, with respect to different pH and different stabilizers.

FIG. 9 illustrates stability of the tanfanercept-containing ophthalmic composition after 4 weeks of a storage at 40° C., in terms of change in IEX-HPLC main peak, with respect to different pH and different stabilizers.

FIG. 10 illustrates stability of the tanfanercept-containing ophthalmic composition after 4 weeks of a storage at 40° C., in terms of change in IEX-HPLC acidic variant, with respect to different pH and different stabilizers.

FIG. 11 illustrates stability of the tanfanercept-containing ophthalmic composition after 4 weeks of a storage at 40° C., in terms of change in IEX-HPLC basic variant, with respect to different pH and different stabilizers.

FIGS. 12 to 14 illustrate results of SEC-HPLC analysis of four (4) types of tanfanercept-containing ophthalmic compositions during storage at 4° C., 25° C. and 40° C., respectively.

FIGS. 15 to 17 illustrate results of acidic variant assay of 4 types of tanfanercept-containing ophthalmic compositions through IEX-HPLC during storage at 4° C., 25° C. and 40° C., respectively.

FIGS. 18 to 20 illustrate results of basic charge variant assay of 4 types of tanfanercept-containing ophthalmic compositions through IEX-HPLC during storage at 4° C., 25° C. and 40° C., respectively.

FIG. 21 illustrates stability of the tanfanercept-containing ophthalmic composition on the basis of aggregate assay through SEC-HPLC.

FIGS. 22 to 23 illustrate stability of the tanfanercept-containing ophthalmic composition on the basis of charge variant assay through IEX-HPLC.

FIG. 24 schematically illustrates a clinical trial schedule with regard to the tanfanercept-containing ophthalmic composition of the present invention used to patients having dry eye syndrome as the subject to be tested.

FIG. 25 schematically illustrates a method for evaluation of corneal staining scores.

FIG. 26 illustrates changes in corneal staining scores (CSS) of inferior, superior and central corneal regions at the end of administration (8 weeks), as well as the sum of the above CSS values, that is, total CSS, relative to a baseline of intent-to-treat population (ITT population).

FIG. 27 illustrates average change in CCSS of total subjects for 8 weeks.

FIG. 28 illustrates average change in TCSS of total subjects for 8 weeks.

FIG. 29 illustrates average change from the baseline in EDS of the subjects having experience using artificial tears within 1 month from 1^{st} visit at 8 weeks.

FIG. 30 illustrates average change from the baseline in CCSS at 8 weeks with respect to disease severity.

FIG. 31 illustrates average change from the baseline in TCSS at 8 weeks with respect to disease severity.

### DETAILED DESCRIPTION OF PREFERRED EMBODIMENTS OF INVENTION

Advantages and features of the present invention and methods for accomplishing the same will be clarified with reference to the following preparative examples, illustrative examples and experimental examples. However, these examples are provided only to facilitate understanding of the present invention and are not to be construed as limiting the invention.

[Part A] Tanfanercept-Containing Ophthalmic Composition

### Experimental Example 1: Assay of Tanfanercept Charge Variant

Generation of charge variants may influence drug activity, stability and safety and, therefore, the present inventors first analyzed the charge variants of tanfanercept. More particularly, after storing tanfanercept at 37° C. for 4 weeks, isoelectric focusing (IEF) and IEX-HPLC analysis were implemented.

Isoelectric Focusing Method

10 μg per well was loaded in a gel at pH 3.0 to pH 7.0, followed by conducting electrophoresis at 100V for 1 hour, 200V for 1 hour and then 500V for 30 minutes. The product was fixed with 12% trichloroacetic acid for 30 minutes and then stained with Coomassie blue.

IEX-HPLC (Ion Exchange-High Performance Liquid Chromatography)

Ion exchange-high performance liquid chromatography (IEX-HPLC) is used to separate protein using an ion exchanger on the basis of affinity to a fixed phase in a column, which is relevant to net charge of the protein. The present experimental method was conducted using a cation exchange column, a temperature controller (set to 25° C.), an automatic sampler (set to 4° C.), a UV detector driven at 280 nm and a high performance liquid chromatography (HPLC) system that may maintain a flow rate of 0.7 mL/min.

Separation and Purification of Charge Variants

In order to separate and analyze the charge variants present in the tanfanercept-containing ophthalmic composition on the basis of properties thereof, an SP-HP column and a liquid chromatography for separation of protein (FPLC) were used to separate and purify the protein based on a salt concentration. On the basis of charge variant properties, an acidic variant sample A, a main peak sample B and a basic variant sample C were prepared, followed by performing IEF and IEX-HPLC analysis, respectively.

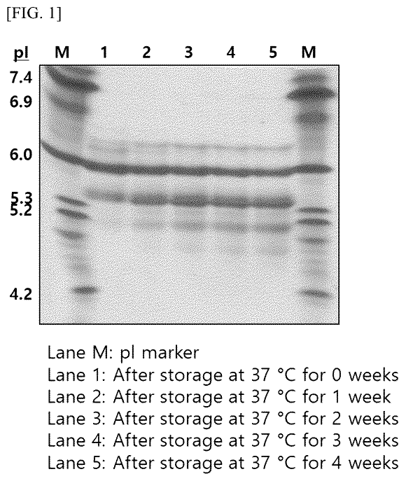

FIG. 1 shows results of isoelectric focusing (IEF) of tanfanercept after storage for 0 to 4 weeks at 37° C.

From the result of IEF, as shown in FIG. 1A, it was obviously confirmed that, as the storage period is extended, the band at a low pI value becomes darker (or thicker), indicating generation of acidic variants of tanfanercept.

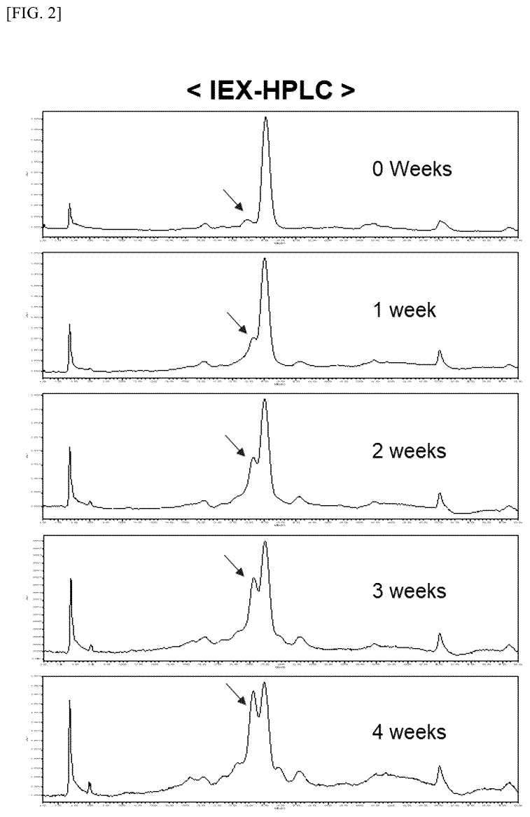

FIG. 2 illustrates results of IEX-HPLC analysis of tanfanercept after storage for 0 to 4 weeks at 37° C.

Further, from the result of IEX-HPLC, as shown in FIG. 2, it was also confirmed that, as the storage period is extended, an amount of acidic variants is increased.

In order to investigate properties of the charge variant, the acidic variant sample, the main peak sample and the basic variant sample obtained by charge-containing separation using an SP-HP column were subjected to IEF and IEX-HPLC analysis.

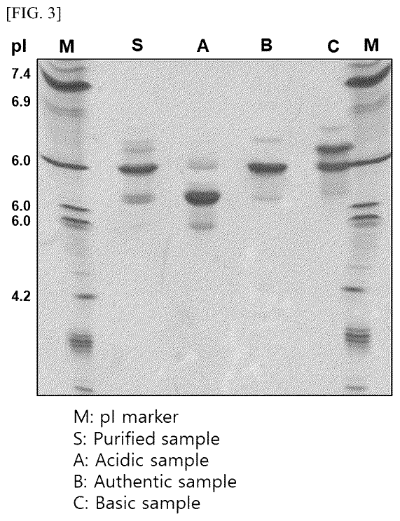

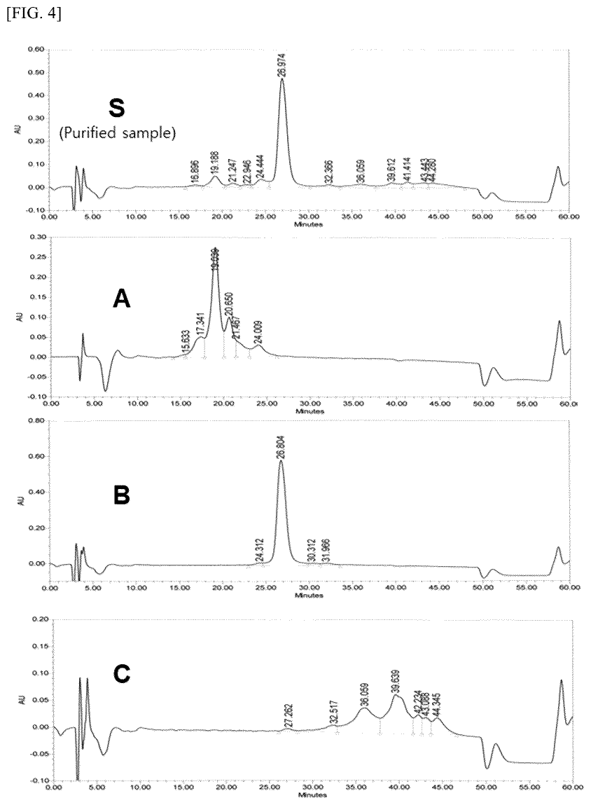

FIG. 3 shows results of IEF analysis of the charge variants. Further, FIG. 4 illustrates results of IEX-HPLC analysis of the charge variants. As shown in FIGS. 3 and 4, the chromatogram result demonstrated that only sample A of the separated acidic variant was eluted before the main peak sample. Also, it could be seen from the IEF result that sample A has a lower pI value than the main peak sample. In addition, the chromatogram result also demonstrated that the basic variant sample, that is, sample B was eluted after the main peak sample. Further, it could be seen from the IEF result that sample B has a slightly higher pI value than the main peak sample.

### Experimental Example 2: Screening of Ophthalmic Stabilizer

A stabilizer added to a protein composition is generally needed to stably store a formulation until the formulation is administered to a patient. In fact, impurities such as aggregates or charge variants possibly generated during storage can be considerably reduced using the stabilizer, so as to maintain a stable formulation during storage. Therefore, it is absolutely important to select a desirable stabilizer that can stabilize major components of a protein composition, thereby preparing a stable composition. In order to perform stress experiments of stabilizers (40° C., stored for 4 weeks) and select desired types of the stabilizers that can stabilize tanfanercept as the major component in the present invention, the present inventors first conducted the following experiments.

1) Preparation of Tanfanercept Solution Sample

10 mg/mL of tanfanercept solution in 20 mM sodium citrate buffer containing 125 mM sodium chloride was prepared.

2) Preparation of Citrate Phosphate Buffer pH 7.0

0.37 g of citric anhydride and 2.58 g of disodium hydrophosphate were added to 900 mL of ultrapure water, followed by thoroughly mixing the same. After titration at pH 7.0 using 37% hydrochloric acid or 40% sodium hydroxide, ultrapure water was added to prepare 1 L of final product.

3) Preparation of Stabilizer Screening Solution

Four (4) types of stabilizers (0.149 g of methionine, 0.751 g of glycine, 1.55 g of histidine hydrochloride and 6.84 g of sucrose), respectively, were added to 100 mL of the buffer prepared in item 2), thereby preparing four (4) types of stabilizer-screening compositions at pH 7.0.

TABLE 1

| | | | | Histidine | |
|---|---|---|---|---|---|
| | Control | Methionine | Glycine | hydrochloride | Sucrose |
| pH 7.0, | — | — | — | — | — |
| citrate | — | 100 mM | — | — | — |
| phosphate | — | — | 100 mM | — | — |
| buffer | — | — | — | 100 mM | — |
| | — | — | — | — | 200 mM |

4) Preparation and Assessment of Sample

After adding 10 mL of the solution for screening ophthalmic stabilizer, which was prepared in item 3), along with tanfanercept to 3.5 kDa centrifugation filter, the sample was centrifuged at 4° C. and 4000 rpm so as to substitute the stabilizer-screening buffer in item 3) for the tanfanercept buffer in item 1). The above procedure was repeated to produce a stabilizer-screening solution containing 1 mg/mL of tanfanercept (sample). The resultant sample was stored at 40° C. for 4 weeks, followed by analyzing the sample through IEX-HPLC at 0 weeks and 4 weeks, respectively, so as to analyze physicochemical impurities identified in each sample.

TABLE 2

| | IEX-HPLC | | | | | | | | |
|---|---|---|---|---|---|---|---|---|---|
| | Start point | | | 4 weeks | | | Change (% peak area) | | |
| | Acidic | Main | Basic | Acidic | Main | Basic | Acidic | Main | Basic |
| Stabilizer | variant | peak | variant | variant | peak | variant | variant | peak | variant |
| Without | 0.7 | 97.7 | 1.6 | 34.7 | 50.8 | 14.6 | 33.9 | −46.9 | 13.0 |
| stabilizer | | | | | | | | | |
| Methionine | 0.7 | 98.3 | 1.0 | 35.4 | 50.0 | 14.7 | 34.7 | −48.3 | 13.7 |
| Glycine | 1.0 | 97.1 | 2.0 | 35.6 | 50.2 | 14.2 | 34.7 | −46.9 | 12.2 |
| Sucrose | 0.8 | 97.9 | 1.3 | 32.3 | 54.9 | 12.9 | 31.5 | −43.0 | 11.6 |
| Histidine | 0.9 | 97.6 | 1.5 | 24.2 | 62.4 | 13.4 | 23.3 | −35.2 | 11.9 |
| hydrochloride | | | | | | | | | |

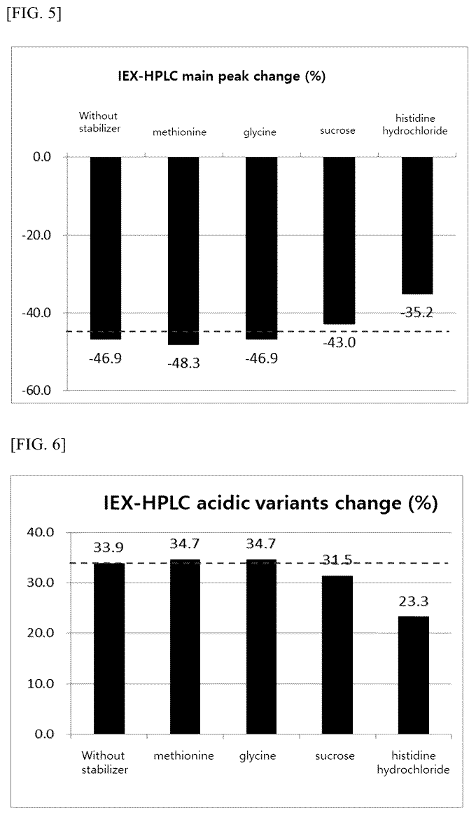

Table 2 and FIGS. 5 to 7 showed IEX-HPLC analysis results of the control group without any stabilizer and the stabilizer screening groups which include methionine, glycine, histidine hydrochloride and sucrose, respectively, as a stabilizer.

Therefore, as shown in Table 2 and FIGS. 5 to 7, it was confirmed that inclusion of sucrose and histidine hydrochloride shows a tendency of generation of basic variants and acidic variants to be reduced. Specifically, in the case of histidine hydrochloride, it could be seen that generation of the acidic variants is significantly reduced as compared to the control group or other stabilizers.

### Experimental Example 3: Preparation of Ophthalmic Composition at Different pH and Assessment of Stability Thereof

Since tears have pH 7.0 to 7.5, it is most preferable to set similar pH conditions for an ophthalmic composition. However, pH may also greatly influence protein stability. Therefore, it was intended to prepare ophthalmic compositions with different pH values and assess stability thereof.

(1) Preparation of Ophthalmic Composition

1) Preparation of 20 mM Citrate Phosphate Buffer at pH 5.0 to pH 7.0

Citric anhydride and disodium hydrophosphate were added to 400 mL of ultrapure water to correspond to desired pH (in case of pH 5.0/5.5 buffer: 0.62 g of citric anhydride and 0.97 g of disodium hydrophosphate; in case of pH 6.0/6.5 buffer: 0.43 g of citric anhydride and 1.11 g of disodium hydrophosphate; in case of pH 7.0 buffer: 0.19 g of citric anhydride and 1.29 g of disodium hydrophosphate). Further, after titration to pH 5.0 to pH 7.0 using 37% hydrochloric acid or 40% sodium hydroxide in order to correspond to different conditions, ultrapure water was added to prepare 500 mL of final product.

2) Preparation of Stabilizer-Added Ophthalmic Composition (pH 5.0 to pH 7.0)

To 100 mL of each of five (5) buffers at pH 5.0 to pH 7.0 prepared in item 1), 6.85 g of sucrose and 1.55 g of histidine were added, thereby preparing five (5) types of 20 mM citrate phosphate buffers at pH 5.0 to pH 7.0, each of which contains 200 mM sucrose, as well as five (5) types of 20 mM citrate phosphate buffers at pH 5.0 to pH 7.0, each of which contains 100 mM histidine. As stabilizer-free experimental groups, the five (5) 20 mM citrate phosphate buffers at pH 5.0 to pH 7.0 prepared in item 1) as described above were directly used as stabilizer-free experimental groups. Therefore, a total of 15 types of buffers at pH 5.0 to pH 7.0 with/without stabilizers was prepared.

3) Preparation and Assessment of Sample

After adding 4 mL of each of the buffers prepared in item 2) as well as tanfanercept, the sample was subjected to centrifugation using a 3.5 kDa centrifugal filter, followed by replacing the existing tanfanercept buffer with the buffer containing a stabilizer at pH 5.0 to pH 7.0. The above procedures were repeated to obtain 15 types of samples containing different stabilizers and having different pH values. The prepared samples were stored at 40° C. for 4 weeks and then analyzed at 0 weeks and 4 weeks, so as to analyze physicochemical impurities identified in each sample.

TABLE 3
Preparation of stabilizer-added ophthalmic
composition (pH 5.0 to pH 7.0)

| | Without stabilizer | Histidine | Sucrose |
|---|---|---|---|
| pH 5.0 | 0 mM | 100 mM | 200 mM |
| pH 5.5 | 0 mM | 100 mM | 200 mM |
| pH 6.0 | 0 mM | 100 mM | 200 mM |
| pH 6.5 | 0 mM | 100 mM | 200 mM |
| pH 7.0 | 0 mM | 100 mM | 200 mM |

(2) Assessment of Stability of Tanfanercept to pH and Stabilizer

1) Reverse Phase Chromatography Analysis

RP-HPLC (reverse phase chromatography) is a method for evaluating purity based on polarity of proteins. The present experimental method was conducted using a reverse phase chromatography column, a temperature controller (set to 60° C.), an automatic sampler (set to 4° C.), a UV detector driven at 214 nm and a high performance liquid chromatography (HPLC) system that may maintain a flow rate of 1.0 mL/min.

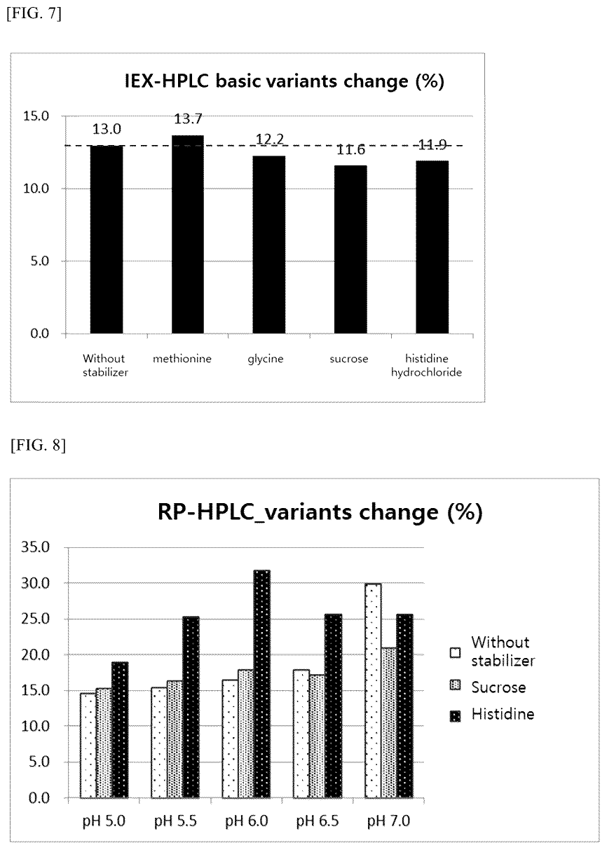

The prepared 15 types of samples were stored under stress conditions (stored at 40° C.) for 4 weeks and then analyzed at 0 weeks and 4 weeks, so as to analyze a change in generation of variants identified in each of the samples. Therefore, as shown in Table 4 and FIG. 8, it was confirmed from the result that the experimental groups at pH 7.0 including sucrose or histidine as a stabilizer exhibit lower rate of generation of variants than the stabilizer-free experimental groups. However, in the case of the experimental groups including histidine as the stabilizer, higher rate of generation of variants at pH 5.0 to pH 6.5 than the control group was demonstrated. Further, in the experimental groups including sucrose as the stabilizer, higher rate of generation of variants at pH 5.0 to pH 6.0 than the control group was demonstrated.

TABLE 4

| Reverse phase | RP-HPLC_Change of variant (% peak area) | | | | |
|---|---|---|---|---|---|
| chromatography | pH 5.0 | pH 5.5 | pH 6.0 | pH 6.5 | pH 7.0 |
| Without | 14.55 | 15.32 | 16.32 | 17.8 | 29.86 |
| stabilizer | | | | | |
| Sucrose | 15.28 | 16.33 | 17.86 | 17.18 | 20.93 |
| Histidine | 18.89 | 25.26 | 31.69 | 25.54 | 25.65 |

2) Ion Exchange High Performance Liquid Chromatography (IEX-HPLC) Analysis

IEX-HPLC is used to separate protein using an ion exchanger on the basis of affinity to a fixed phase of a column due to net charge of the protein. The present experimental method was conducted using a cation exchange column, a temperature controller (set to 25° C.), an automatic sampler (set to 4° C.), a UV detector driven at 280 nm and a high performance liquid chromatography (HPLC) system that may maintain a flow rate of 0.7 mL/min.

The prepared 15 types of samples were stored under stress conditions (stored at 40° C.) for 4 weeks and then analyzed at 0 weeks and 4 weeks, followed by comparing changes in generation of acidic/basic variants identified in each of the samples.

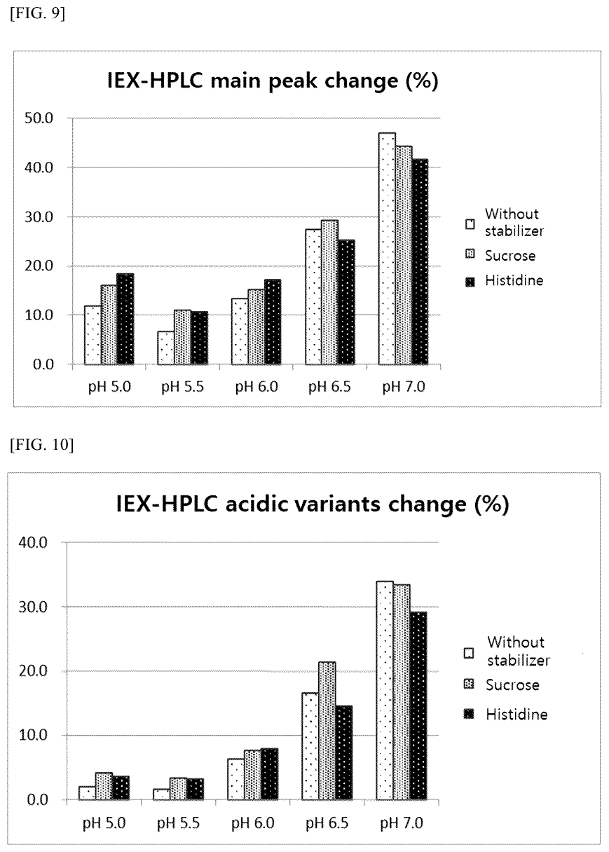

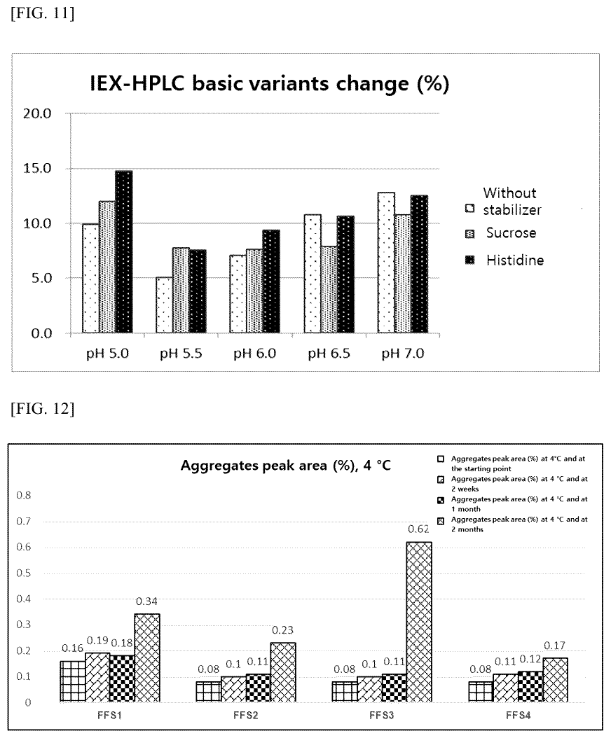

Therefore, as shown in Table 5 and FIGS. 9 to 11, it could be seen that the stabilizer-free group at pH 5.0 to pH 6.5 exhibits better results than the experimental groups containing sucrose or histidine in terms of generation of acidic variants. Specifically, the stabilizer-free group at pH 5.0 to pH 6.0 showed less than 10% of change in generation of acidic variants. For basic variants, the stabilizer-free group at pH 5.0 to pH 6.0 showed better results than the experimental groups containing sucrose or histidine. At pH 6.5 to pH 7.0, the experimental group containing sucrose showed the lowest change in generation of basic variants while the experimental group containing histidine did not have a significant difference from the stabilizer-free group.

TABLE 5

| | Change of acidic variant | | | | | Change of basic variant | | | | | Change of main peak | | | | |
|---|---|---|---|---|---|---|---|---|---|---|---|---|---|---|---|
| | (% peak area) | | | | | (% peak area) | | | | | (% peak area) | | | | |
| IEX- | pH | pH | pH | pH | pH | pH | pH | pH | pH | pH | pH | pH | pH | pH | pH |
| HPLC | 5.0 | 5.5 | 6.0 | 6.5 | 7.0 | 5.0 | 5.5 | 6.0 | 6.5 | 7.0 | 5.0 | 5.5 | 6.0 | 6.5 | 7.0 |
| Without | 2.00 | 1.60 | 6.30 | 16.60 | 34.00 | 9.90 | 5.10 | 7.10 | 10.80 | 12.80 | 11.90 | 6.70 | 13.40 | 27.40 | 46.90 |
| stabilizer | | | | | | | | | | | | | | | |
| Sucrose | 4.06 | 3.28 | 7.60 | 21.31 | 33.41 | 12.02 | 7.77 | 7.60 | 7.87 | 10.80 | 16.08 | 11.06 | 15.21 | 29.19 | 44.21 |
| Histidine | 3.54 | 3.15 | 7.83 | 14.55 | 29.07 | 14.76 | 7.58 | 9.40 | 10.67 | 12.56 | 18.30 | 10.73 | 17.24 | 24.23 | 41.62 |

As a result of analyzing the above measured values, the experimental group containing sucrose or histidine at pH 7.0 showed a tendency of less generation of variants than the stabilizer-free group. On the contrary, when the pH value is decreased to pH 5.5 or pH 6.0, it could be seen that the generation of variants in the experimental group containing sucrose or histidine tends to significantly increase, as compared to the stabilizer-free group.

Combining the experimental results, it could be determined that, contrary to expectations, the experimental group of the tanfanercept-containing ophthalmic composition without any stabilizer at pH 5.5 to pH 6.0, that is, the stabilizer-free group exhibits lowest generation of variants.

In general, the ophthalmic composition may be set to pH 7.0, which is similar to in vivo pH. However, stability of an active ingredient in the ophthalmic composition is an essential point for expressing medical efficacy, thereby coming to the conclusion that the tanfanercept-containing ophthalmic composition of the present invention is preferably set to have pH 5.5 to pH 6.0 while not containing sucrose or histidine.

### Experimental Example 4: Assessment of Stability to Buffer System

In order to compare differences in constitutional composition of buffers and addition/non-addition of two functional excipients while fixing a concentration of tanfanercept, pH, a concentration of sodium chloride, osmotic pressure, etc., four (4) experimental groups were prepared and screened at 4° C., 25° C. and 40° C., respectively, so as to select a final formulation. pH of the ophthalmic composition was fixed to pH 5.5 in consideration of the experimental results described above. Further, as a normal buffer generally useable in the above range, sodium acetate (20 mM) and sodium citrate (20 mM) were used to assess tanfanercept relative to the buffer system.

As shown in Table 6 below, four (4) types of tanfanercept-containing ophthalmic compositions were prepared and used to conduct an experiment for stability of tanfanercept.

TABLE 6

| Component | FFS1 | FFS2 | FFS3 | | FFS4 | |
|---|---|---|---|---|---|---|
| Tanfanercept | 6.25 | | mg/mL | | | |
| pH | 5.5 | | | | | |
| Sodium acetate | 1.44 g | — | — | | — | |
| anhydride | | | | | | |
| Acetic acid | 0.145 g | — | — | | — | |
| Sodium citrate | — | 4.75 g | 4.75 | g | 4.75 | g |
| dianhydride | | | | | | |
| Citric acid | — | 0.75 g | 0.75 | g | 0.75 | g |

| | | | | | |
|---|---|---|---|---|---|
| Sodium hyaluronate | — | — | 0.2 | mL | — |

| | | | | |
|---|---|---|---|---|
| Hypromellose | — | — | 0.8 | mL |

| | | |
|---|---|---|
| Sodium chloride | 6.428 | g |
| Ultrapure water | 1 | L |

By means of analysis of aggregates by SEC-HPLC and analysis of charge variants, the tanfanercept-containing ophthalmic composition was subjected to a stability experiment. The stability experiment was conducted at 4° C., 25° C. and 40° C. for 2 months in total. At the time of 0 weeks, 2 weeks, 1 month and 2 months, aggregate analysis through SEC-HPLC and charge variant analysis through IEX-HPLC were conducted.

(1) Aggregates analysis by SEC-HPLC

Size-exclusion high performance liquid chromatography (SEC-HPLC) is typically used to separate protein on the basis of size difference by introducing a fixed phase sample into a column filled with a porous gel. The present experimental method was conducted using a size-exclusion column, a temperature controller (set to 25° C.), an automatic sampler (set to 4° C.), a UV detector driven at 214 nm and a high performance liquid chromatography (HPLC) system that may maintain a flow rate of 0.5 mL/min.

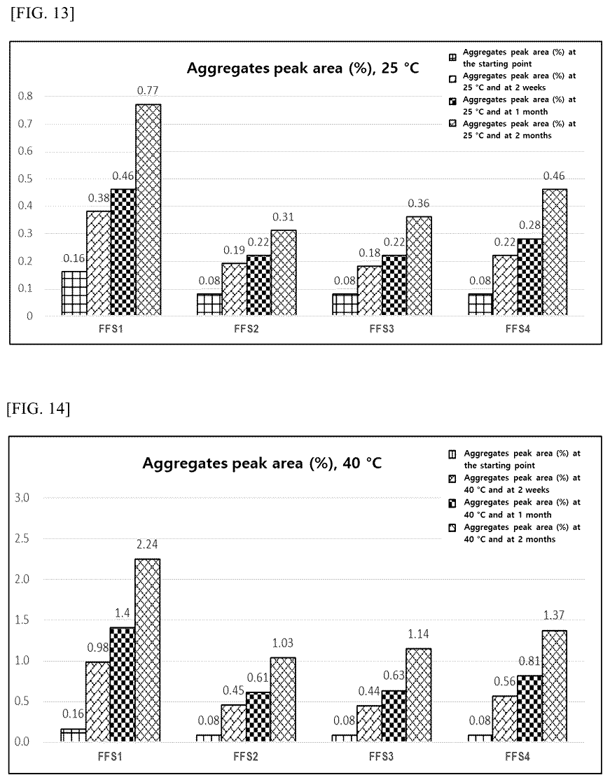

Tables 7 to 9, and FIGS. 12 to 14 demonstrated results of SEC-HPLC analysis that was performed while storing the above four (4) types of tanfanercept-containing ophthalmic compositions at 4° C., 25° C. and 40° C. From SEC-HPLC analysis results, it could be seen that the higher the temperature, the greater the concentration of aggregates. Specifically, the aggregates were rapidly generated in case of the formulation including a buffer. Among three (3) types of formulations prepared with a citrate buffer, FFS3 was measured to have a high content of aggregates after storage at 4° C. for 2 months. Further, FFS2 without any excipient showed the most stable results in all conditions including 25° C., 40° C., 2 weeks, 1 month and 2 months.

TABLE 7
SEC aggregates analysis [4° C.]

| | | Peak | | | |
|---|---|---|---|---|---|
| | Type of | % peak area of aggregates | | | |
| No. | sample | 0 | 0.5 | 1 | 2 |
| 1 | FFS1 | 0.16 | 0.19 | 0.18 | 0.34 |
| 2 | FFS2 | 0.08 | 0.10 | 0.11 | 0.23 |
| 3 | FFS3 | 0.08 | 0.10 | 0.11 | 0.62 |
| 4 | FFS4 | 0.08 | 0.11 | 0.12 | 0.17 |

TABLE 8
SEC aggregates analysis [25° C.]

| | |
|---|---|
| Peak | |
| Type of | % peak area of aggregates |

| No. | sample | 0 | 0.5 | 1 | 2 |
|---|---|---|---|---|---|
| 1 | FFS1 | 0.16 | 0.38 | 0.46 | 0.77 |
| 2 | FFS2 | 0.08 | 0.19 | 0.22 | 0.31 |
| 3 | FFS3 | 0.08 | 0.18 | 0.22 | 0.36 |
| 4 | FFS4 | 0.08 | 0.22 | 0.28 | 0.46 |

TABLE 9
SEC aggregates analysis [40° C.]

| | |
|---|---|
| Peak | |
| Type of | % peak area of aggregates |

| No. | sample | 0 | 0.5 | 1 | 2 |
|---|---|---|---|---|---|
| 1 | FFS1 | 0.16 | 0.98 | 1.40 | 2.24 |
| 2 | FFS2 | 0.08 | 0.45 | 0.61 | 1.03 |
| 3 | FFS3 | 0.08 | 0.44 | 0.63 | 1.14 |
| 4 | FFS4 | 0.08 | 0.56 | 0.81 | 1.37 |

(2) Acidic Variants Analysis by IEX-HPLC

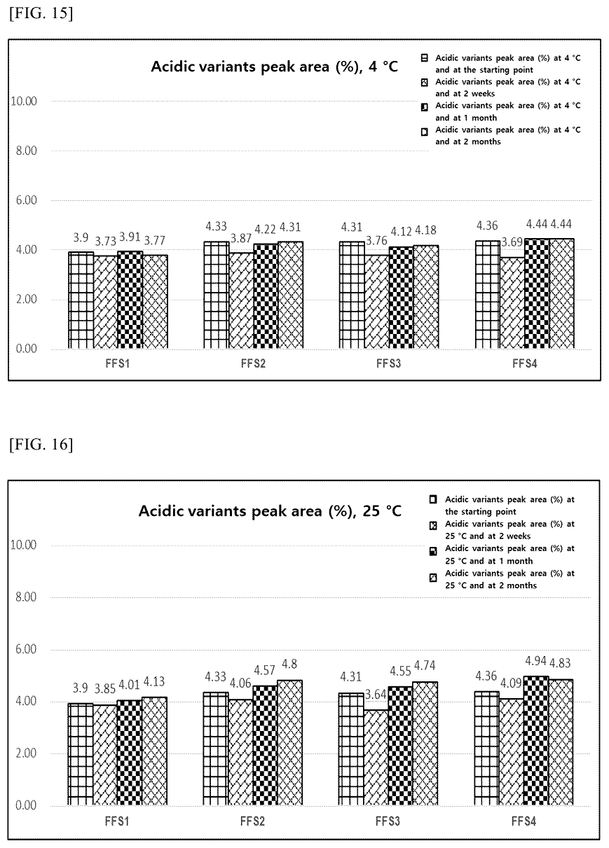

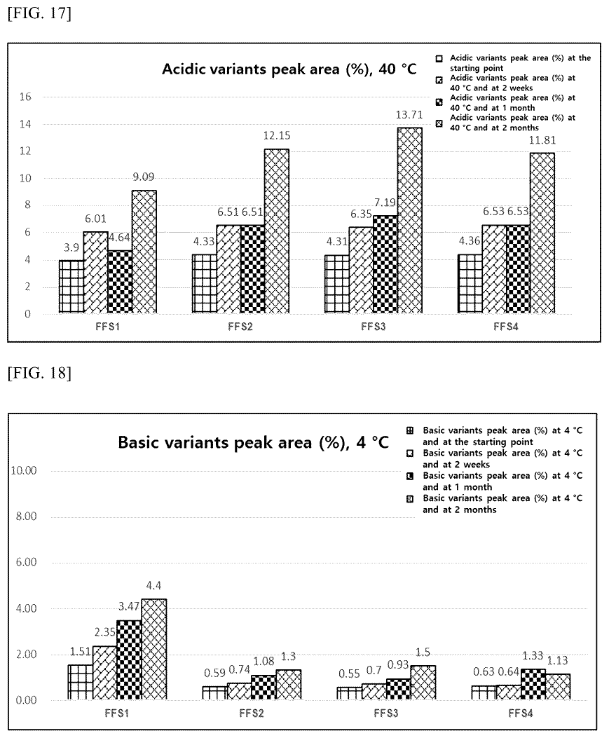

Tables 10 to 12, and FIGS. 15 to 17 demonstrated results of analysis of acidic variants that was performed by IEX-HPLC while storing the above four (4) types of tanfanercept-containing ophthalmic compositions at 4° C., 25° C. and 40° C.

TABLE 10
IEX-acidic variants [4° C.]

| | |
|---|---|
| Peak | |
| Type of | % peak area of acidic variants |

| No. | sample | 0 | 0.5 | 1 | 2 |
|---|---|---|---|---|---|
| 1 | FFS1 | 3.90 | 3.73 | 3.91 | 3.77 |
| 2 | FFS2 | 4.33 | 3.87 | 4.22 | 4.31 |
| 3 | FFS3 | 4.31 | 3.76 | 4.12 | 4.18 |
| 4 | FFS4 | 4.36 | 3.69 | 4.44 | 4.44 |

TABLE 11
IEX-acidic variants [25° C.]

| | |
|---|---|
| Peak | |
| Type of | % peak area of acidic variants |

| No. | sample | 0 | 0.5 | 1 | 2 |
|---|---|---|---|---|---|
| 1 | FFS1 | 3.90 | 3.85 | 4.01 | 4.13 |
| 2 | FFS2 | 4.33 | 4.06 | 4.57 | 4.80 |
| 3 | FFS3 | 4.31 | 3.64 | 4.55 | 4.74 |
| 4 | FFS4 | 4.36 | 4.09 | 4.94 | 4.83 |

TABLE 12
IEX-acidic variants [40° C.]

| | |
|---|---|
| Peak | |
| Type of | % peak area of acidic variants |

| No. | sample | 0 | 0.5 | 1 | 2 |
|---|---|---|---|---|---|
| 1 | FFS1 | 3.90 | 6.01 | 4.64 | 9.09 |
| 2 | FFS2 | 4.33 | 6.51 | 6.51 | 12.15 |
| 3 | FFS3 | 4.31 | 6.35 | 7.19 | 13.71 |
| 4 | FFS4 | 4.36 | 6.53 | 6.53 | 11.81 |

(3) Basic Variants Analysis by IEX-HPLC

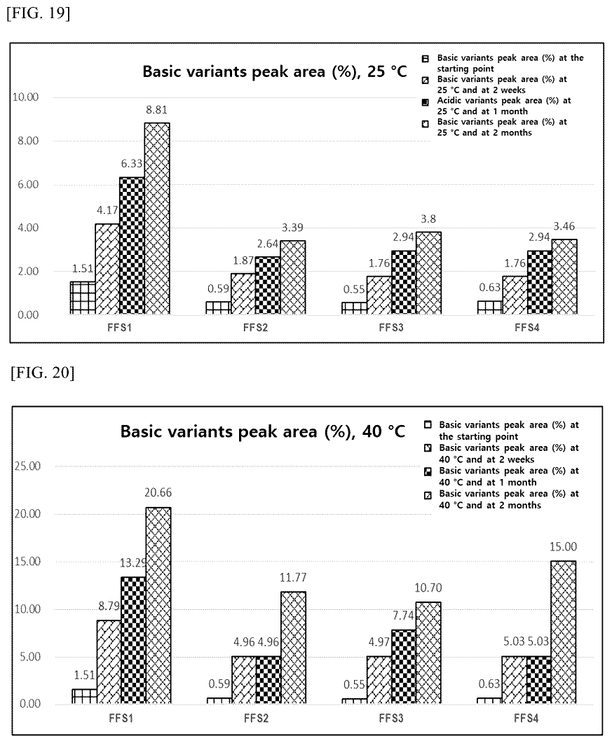

Tables 13 to 15, and FIGS. 18 to 20 demonstrated results of analysis of basic variants that was performed by IEX-HPLC while storing the above four (4) types of tanfanercept-containing ophthalmic compositions at 4° C., 25° C. and 40° C.

TABLE 13
IEX-basic variants [4° C.]

| | |
|---|---|
| Peak | |
| Type of | % peak area of basic variants |

| No. | sample | 0 | 0.5 | 1 | 2 |
|---|---|---|---|---|---|
| 1 | FFS1 | 1.51 | 2.35 | 3.47 | 4.40 |
| 2 | FFS2 | 0.59 | 0.74 | 1.08 | 1.30 |
| 3 | FFS3 | 0.55 | 0.70 | 0.93 | 1.50 |
| 4 | FFS4 | 0.63 | 0.64 | 1.33 | 1.13 |

TABLE 14
IEX-basic variants [25° C.]

| | |
|---|---|
| Peak | |
| Type of | % peak area of basic variants |

| No. | sample | 0 | 0.5 | 1 | 2 |
|---|---|---|---|---|---|
| 1 | FFS1 | 1.51 | 4.17 | 6.33 | 8.81 |
| 2 | FFS2 | 0.59 | 1.87 | 2.64 | 3.39 |
| 3 | FFS3 | 0.55 | 1.76 | 2.94 | 3.80 |
| 4 | FFS4 | 0.63 | 1.76 | 2.94 | 3.46 |

TABLE 15
IEX-basic variants [40° C.]

| | |
|---|---|
| Peak | |
| Type of | % peak area of acidic variants |

| No. | sample | 0 | 0.5 | 1 | 2 |
|---|---|---|---|---|---|
| 1 | FFS1 | 1.51 | 8.79 | 13.29 | 20.66 |
| 2 | FFS2 | 0.59 | 4.96 | 4.96 | 11.77 |
| 3 | FFS3 | 0.55 | 4.97 | 7.74 | 10.70 |
| 4 | FFS4 | 0.63 | 5.03 | 5.03 | 15.00 |

From IEX-HPLC analysis, it could be seen that all formulations show a tendency of acidic and basic variants to be increased under storage conditions at a high temperature of 40° C., specifically, the acetate buffer (FFS1) formulation exhibits a pattern of significantly increasing the basic variant at all temperatures.

Consequently, in an aspect of generation of aggregates and basic variants, it was confirmed that stability of the acetate buffer (FFS1) is more deteriorated than the citrate buffers (FFS2 to 4). Further, on the basis of constitutional composition of the citrate buffer, when comparing functional excipient-added groups with a group without functional excipient, the group without functional excipient was proved to be most stable. Therefore, in overall consideration of simplification of a product manufacturing process, contamination in the manufacturing process and uniformity, a formulation using the citrate buffer (FFS2) was adopted as a final formulation. However, considering that physiological pH of tears is neutral, pH 6.0 at which patient compliance may be a little higher along with an acceptable level of stability was selected as pH of the final product.

### Preparative Example 1: Production of Tanfanercept-Containing Ophthalmic Composition

To 900 ml of ultrapure water, 5.35 g of trisodium citrate dihydrate, 0.35 g of citric acid anhydride and 7.3 g of sodium chloride were added and completely dissolved, thereby preparing a buffer. After confirming pH of the buffer as pH 6.0±0.1, an amount of the buffer was adjusted to reach a final solution volume of 1 L using a graduated cylinder and then filtered through a 0.22 μm bottle top filter system.

Then, tanfanercept was added to the prepared buffer to produce 0.25% tanfanercept-containing eye drop composition.

TABLE 16

| Component | Content (mg/ml) |
|---|---|
| Tanfanercept | 2.5 |
| Trisodium citrate, dihydrate | 5.35 |
| Citric acid anhydride | 0.35 |
| Sodium chloride | 7.31 |
| Sodium hydroxide | As required |
| Hydrochloric acid | As required |
| Ultrapure water | Q.S. |

### Experimental Example 5: Assessment of Stability of Tanfanercept-Containing Ophthalmic Composition

The tanfanercept-containing ophthalmic composition according to Preparative Example 1 was subjected to assessment of stability.

A long term storage condition was set to 5° C. (humidity not adjusted), and an accelerated condition was set to 25° C./60% RH. The composition in Preparative Example 1 was stored under these conditions, and then, was subjected to aggregate analysis through SEC-HPLC and charge variant analysis through IEX-HPLC.

TABLE 17

| Analysis | Storage | | 3 | 6 | 9 | 12 | 18 | 24 | 36 |
|---|---|---|---|---|---|---|---|---|---|
| method | condition | Start | months | months | months | months | months | months | months |
| SEC-HPLC | Long term | 0.1 | 0.2 | 0.1 | 0.2 | 0.2 | 0.2 | 0.2 | 0.3 |
| (aggregate, %) | storage | | | | | | | | |
| | Accelerated | 0.1 | 0.3 | 0.4 | | | | | |

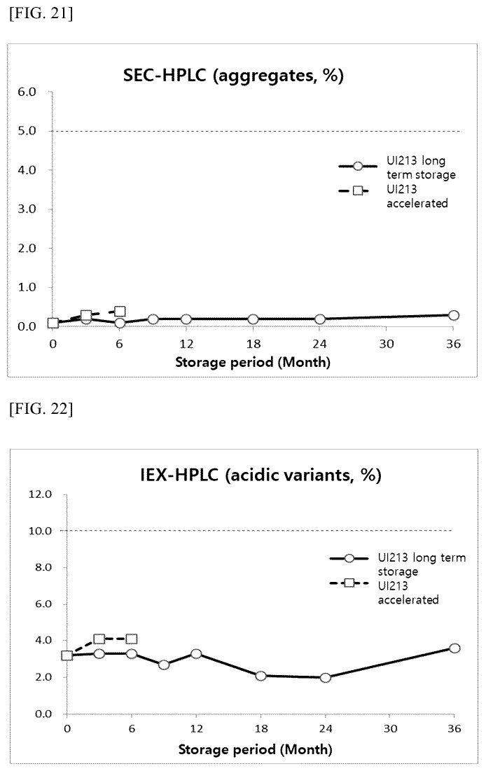

Table 17 and FIG. 21 showed results of the aggregate analysis through SEC-HPLC. As shown in Table 17 and FIG. 21, the composition in Preparative Example 1 was confirmed to be stable since aggregates were formed within 5% when the composition was stored for 3 years under a long term storage condition at 5° C. Further, when the composition was stored for 6 months under accelerated conditions at 25° C./60% RH, generation of aggregates within 5% was demonstrated.

TABLE 18

| Analysis | Storage | | 3 | 6 | 9 | 12 | 18 | 24 | 36 |
|---|---|---|---|---|---|---|---|---|---|
| method | condition | Start | months | months | months | months | months | months | months |
| IEX- | Long term | 3.2 | 3.3 | 3.3 | 2.7 | 3.3 | 2.1 | 2 | 3.6 |
| HPLC | storage | | | | | | | | |
| (acidic | | | | | | | | | |
| variant, | Accelerated | 3.2 | 4.1 | 4.1 | | | | | |
| %) | | | | | | | | | |
| IEX- | Long term | 1.1 | 1.5 | 1.4 | 2.6 | 1.9 | 3.2 | 5.1 | 8 |
| HPLC | storage | | | | | | | | |
| (basic | Accelerated | 1.1 | 3.7 | 5.9 | | | | | |
| variant, | | | | | | | | | |
| %) | | | | | | | | | |

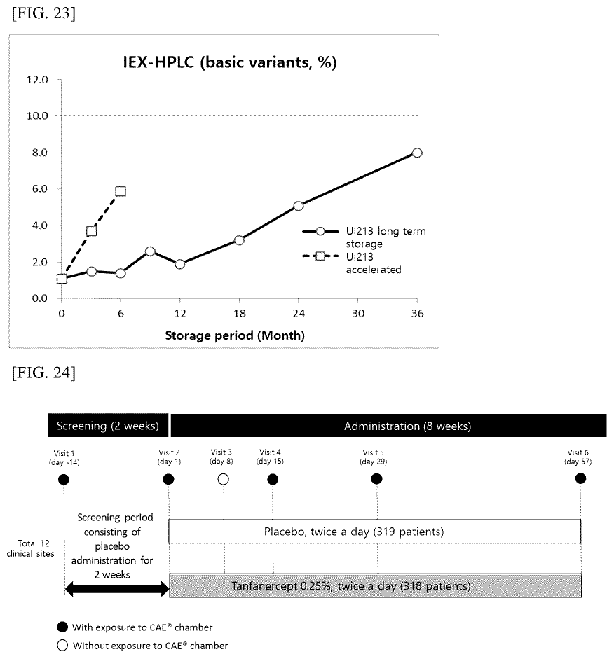

Table 18 and FIGS. 22 to 23 show results of charge variant analysis through IEX-HPLC. As shown in Table 18 and FIGS. 22 to 23, the composition in Preparative Example 1 was confirmed to be stable since charge variants were formed within 5% during storage when the composition was stored for 3 years under a long term storage condition at 5° C. Further, the basic variants were also formed within 10%, thereby demonstrating stability during storage.

In addition, when the composition was stored for 6 months under accelerated conditions at 25° C./60% RH, formation of acidic variants within 10% was demonstrated. Further, the basic variants were formed within 10%, thereby demonstrating stability during storage.

[Part B] Clinical Trial for Tanfanercept-Containing Ophthalmic Composition

In order to assess safety and efficacy of tanfanercept-containing ophthalmic composition relative to placebo, the following clinical trial was conducted.

Procedures of Clinical Trial

To subjects, that is, patients suffering from dry eye syndrome, a Phase 3, multicenter, randomized, double-masked and placebo-controlled clinical trial was performed. After administering eye drops to 637 patients twice a day for 8 weeks, inferior corneal staining score (hereinafter abbrev. to “ICSS”) and ocular discomfort score (hereinafter abbrev. to “ODS”) as primary endpoints, as well as central corneal staining score (hereinafter abbrev. to “CCSS”), total corneal staining score (hereinafter abbrev. to “TCSS”) and eye dryness score (hereinafter abbrev. to “EDS”) as secondary endpoints were evaluated.

1. Schedule of Clinical Trial

The clinical trial has proceeded in the order illustrated in FIG. 24. The subjects visited a clinical trial sites a total 6 of visits during a clinical trial period of 10 weeks including a 2-week screening process followed by a 8-week dosing period. First screening was conducted at the first visit, and second screening was conducted at the second visit. The subjects who passed both screening processes were subjected to dosing by the following dosing method.

2. Screening Method

In a total of 12 clinical trial sites, screening was implemented for a total of 1109 subjects. There among, 472 subjects were excluded during screening, and the remaining 637 subjects were dosed. During screening, the subjects were exposed to a controlled adverse environment (CAE®) chamber twice for 90 minutes (Visit 1, Visit 2). Selection criteria for the subjects are as follows.

Screening Criteria on Visit 1:
1) Have a history of dry eye for at least 6 months,
2) Have a history of use or desire to use eye drops for dry eye symptoms within 6 months,
3) Have a best corrected visual acuity of 0.7 log MAR or better.

Screening Criteria on Visit 1 and Visit 2:
1) Report a score of 2 or more according to the Ora Calibra® Ocular Discomfort & 4-Symptom Questionnaire,
2) Have a Schirmer's Test score of 1 mm to 10 mm,
3) Have a corneal staining score (CSS) of 2 or more in at least one region,
4) Have a conjunctival redness score of 1 or more,
5) Have at least 1 point or more increase in ICSS (inferior corneal staining score) following CAE exposure. Additionally, while ODS was measured at an interval of 5 minutes during the 90 minutes of CAE exposure, a score of 3 or greater must be reported at two or more consecutive time points during CAE exposure.

Have the same eye, satisfy all criteria for 2) to 5).

On Visit 2, the finally qualified subjects were randomized blindly to test drug (tanfanercept) or placebo group.

3. Patient Groups

637 subjects who passed through the screening process participated in the clinical trial. They were randomly allocated in a double-blind to receive the clinical study treatment. 318 subjects were administered tanfanercept-containing ophthalmic solution (0.25%), while 319 subjects received placebo ophthalmic solution. Demographic information of clinical trial subjects is shown in Table 19 below.

TABLE 19
Demographic information of clinical trial subjects

| | | |
|---|---|---|
| Test drug group | Placebo group | Total subjects |
| (N = 318) | (N = 319) | (N = 637) |

Age (years)

| | | | | | | |
|---|---|---|---|---|---|---|
| Mean (standard | 61.0 | (12.86) | 62.9 | (14.02) | 62.0 | (13.48) |
| deviation) | | | | | | |

| | | | |
|---|---|---|---|
| Minimum, | 19, 89 | 23, 92 | 19, 92 |
| maximum | | | |

Age categories, number (%)

| | | | | | | |
|---|---|---|---|---|---|---|
| <65 years | 175 | (55.0%) | 162 | (50.8%) | 337 | (52.9%) |
| ≥65 years | 143 | (45.0%) | 157 | (49.2%) | 300 | (47.1%) |

Sex, number (%)

| | | | | | | |
|---|---|---|---|---|---|---|
| Male | 88 | (27.7%) | 108 | (33.9%) | 196 | (30.8%) |
| Female | 230 | (72.3%) | 211 | (66.1%) | 441 | (69.2%) |

Ethnicity, number (%)

| | | | | | | |
|---|---|---|---|---|---|---|
| Hispanic or | 24 | (7.5%) | 25 | (7.8%) | 49 | (7.7%) |
| Latino | | | | | | |
| Non- Hispanic | 294 | (92.5%) | 294 | (92.2%) | 588 | (92.3%) |
| or Latino | | | | | | |

Race, number (%)

| American Indian | 0 | | 2 | (0.6%) | 2 | (0.3%) |
|---|---|---|---|---|---|---|
| or | | | | | | |
| Alaska native | | | | | | |
| Asian | 18 | (5.7%) | 19 | (6.0%) | 37 | (5.8%) |
| Black or African | 71 | (22.3%) | 53 | (16.6%) | 124 | (19.5%) |
| American | | | | | | |
| White | 228 | (71.7%) | 238 | (74.6%) | 466 | (73.2%) |
| Multiple | 1 | (0.3%) | 7 | (2.2%) | 8 | (1.3%) |

4. Test Drug and Placebo

As the test drug, tanfanercept-containing ophthalmic composition (0.25%) prepared in Preparative Example 1 was used. The placebo used herein was prepared with the same composition as the test drug except that tanfanercept was not included.

5. Administration and Assessment

TABLE 20

| | Investigational | Concentration of | |
|---|---|---|---|
| Group | Product | tanfanercept | Dose |
| Test drug | Tanfanercept- | 0.25% | Eye drop to both |
| | containing ophthalmic | | eyes, twice a day |
| | composition | | for 8 weeks |
| | | | (57 ± 3 days) |
| Placebo | Placebo ophthalmic | 0% | |
| | composition | | |

Each subject self-administered the test drug or placebo as shown in Table 20 twice a day (BID) over 8 weeks starting from the second visiting day as a first day of administration, and completed assessment of symptoms daily. The subjects visited the clinical trial sites on day 1 (Visit 2), day 8 (Visit 3), day 15 (Visit 4), day 29 (Visit 5) and day 57 (Visit 6) after starting administration. Signs and symptoms were evaluated for these subjects. On day 15 (Visit 4), day 29 (Visit 5) and day 57 (Visit 6) after starting administration, the subjects were exposed to a CAE® chamber, and the assessment of dry eye signs and symptoms were conducted before, during and after the exposure, respectively. In this regard, only on day 8 (Visit 3) after starting administration, signs and symptoms were assessed without exposure to CAE® chamber.

6. Objective Sign Assessment Indicator

As an objective sign assessment indicator of dry eye syndrome, corneal staining score (CSS) evaluation was used. CSS evaluation is a method of observing corneal damage after dropping a dye into the eye, and then scoring the degree of damage.

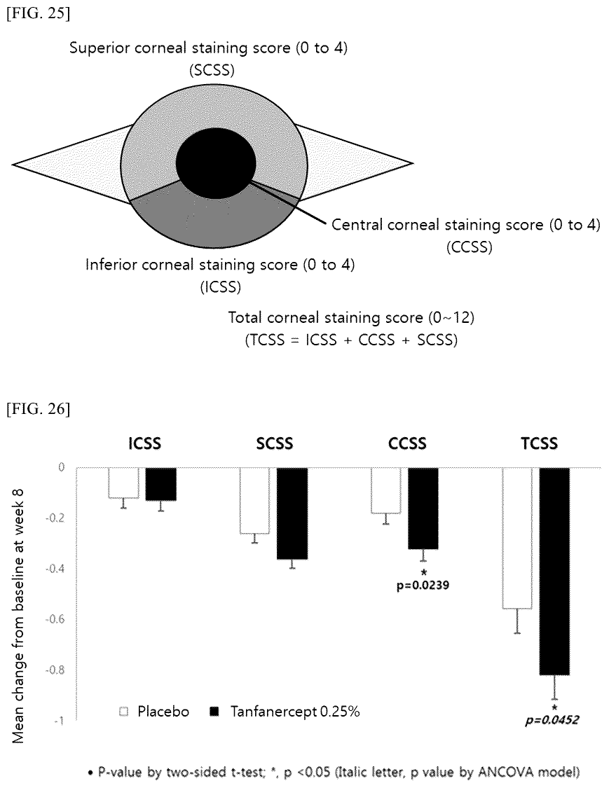

In the present clinical trial, Ora Calibra® corneal staining score scale was used. FIG. 25 schematically illustrates a method for evaluation of corneal staining scores. As shown in FIG. 25, the cornea was divided into the central region (black), upper region (or superior region; bright grey) and lower region (inferior region: dark grey) and staining extents of these portions were evaluated in the range of 0 to 4 points in increments 0.5 points with respect to the standard shown in Table 21 (the higher the score, the more serious the corneal damage). Results of the evaluation were classified into CCSS for the central portion, SCSS for the superior portion and ICS S for the inferior portion, and these values were summed and indicated as TCSS.

TABLE 21
Ora Calibra ® corneal staining score scale

| | | |
|---|---|---|
| 0 | None | No staining |
| 1 | Trace | occasional |
| 2 | Mild | Countable |
| 3 | Moderate | uncountable, but not confluent |
| 4 | Severe | confluent |

7. Subjective Symptom Assessment Indicator

Symptom assessment indicator of dry eye syndrome is improvement of disease symptoms subjectively felt by subjects through questioning, and may be evaluated by ODS, EDS, and other types of questionnaires.

7-1. Ocular Discomfort Score (ODS)

In the present clinical trial, ODS was evaluated by Ora Calibra® ODS.

Extents of ocular discomfort subjectively felt by the subjects, respectively, were scored in the range of 0 to 4 points with respect to the standard shown in Table 22 below (the higher the score, the greater the ocular discomfort).

TABLE 22
Ora Calibra ® ocular discomfort score scale

| | |
|---|---|
| 0 | No discomfort |
| 1 | Intermittent awareness |
| 2 | Constant awareness |
| 3 | Intermittent discomfort |
| 4 | Constant discomfort |

7-2. Eye Dryness Score (EDS)

EDS is a subject's evaluation of eye dryness score on the visual analog scale (VAS). The subjects scored EDS in numbers ranging from 0 to 100. 0 means “no dryness” and 100 indicates “maximum dryness”.

8. Schirmer's Tear Test

Another evaluation method for dry eye syndrome used herein is the Schirmer's tear test. The Schirmer's tear test (STT) is a method in which an STT strip is placed on the brim of the lower eyelid of each eye of a patient, followed by closing the eye for 5 minutes, and then, the STT strip is removed, and a length (mm) of the wet portion is measured and recorded.

Result of Clinical Trial

1. Baseline Assessment

As described in the above clinical trial procedures, on day 1 after starting the clinical trial of the subjects, the objective sign such as CSS and the subjective symptom such as EDS and ODS evaluated before exposure to the CAE® chamber were used as baselines. With regard to 318 subjects given the test drug and 319 patient given placebo, the baseline scores of four (4) types of CSS (ICSS, CCSS, SCSS, TCSS), as well as EDS and ODS are listed in Table 23 below.

TABLE 23
Evaluation of baselines

| Sign or | | Test drug group | | Placebo group | |
|---|---|---|---|---|---|
| symptom | | (N = 318) | | (N = 319) | |
| Sign | ICSS, mean (standard | 1.78 | (0.550) | 1.84 | (0.527) |
| | deviation) | | | | |
| | CCSS, mean (standard | 1.40 | (0.780) | 1.36 | (0.654) |
| | deviation) | | | | |
| | SCSS, mean (standard | 1.81 | (0.617) | 1.82 | (0.556) |
| | deviation) | | | | |
| | TCSS, mean (standard | 4.99 | (1.266) | 5.01 | (1.123) |
| | deviation) | | | | |
| Symptom | EDS, mean (standard | 61.4 | (24.71) | 59.7 | (24.58) |
| | deviation) | | | | |
| | ODS, mean (standard | 2.6 | (0.98) | 2.5 | (1.06) |
| | deviation) | | | | |

2. Result of CSS Evaluation

After completion of clinical drug dosage for 8 weeks, with regard to randomly assigned subjects (intent-to-Treat (ITT) Population), the mean change in CSS from baseline was investigated as the objective sign. FIG. 26 illustrates changes in corneal staining scores (CSS) of inferior, superior and central corneal regions at the end of administration (8 weeks), as well as the sum of the above CSS values, that is, total CSS, relative to a baseline of intent-to-treat population (ITT population, observed data only).

As shown in FIG. 26, the subjects having test drug dosage exhibited significantly improved results of corneal damage relative to placebo at 8 weeks, in terms of central region (CCSS, p=0.024) and total cornea (TCSS, p=0.045).

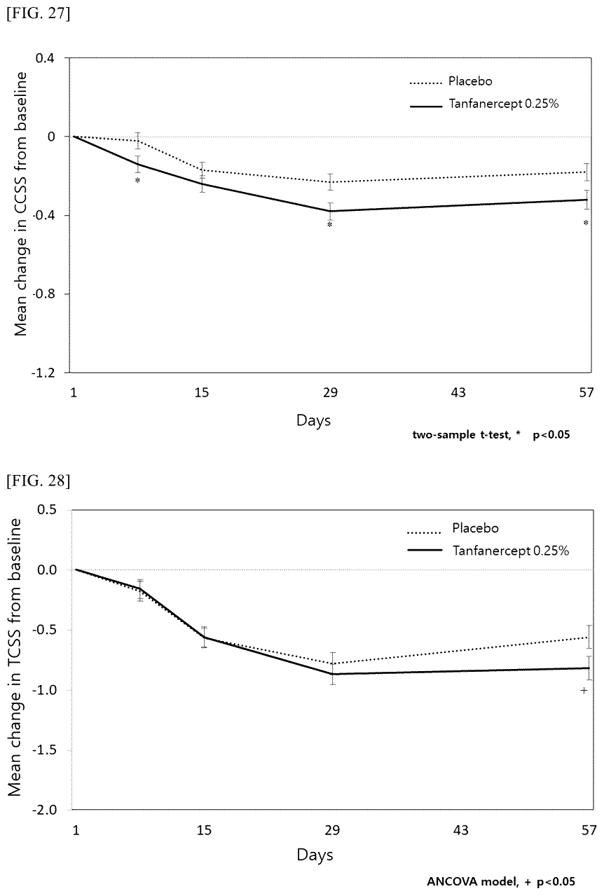

Table 24 and FIGS. 27 to 28 demonstrate changes in CCSS and TCSS over 8 weeks and statistical analysis results of all subjects.

TABLE 24
Mean change in corneal staining score from baseline

| | | | Test drug | Placebo |
|---|---|---|---|---|
| | | | group | group |
| Region | Visit | | (N = 318) | (N = 319) |
| CCSS | Week 1 | Number of subjects | 314 | 314 |
| | Visit 3 | Mean (standard | −0.140 | −0.02 |
| | (8 ± 1 days) | deviation) | (0.726) | (0.746) |
| | | Least squares (LS) | −0.09 | |
| | | mean difference | | |
| | | Significant | 0.0562 | |
| | | probability | | |
| | | (p-value), | | |
| | | covariance | | |
| | | (ANCOVA) | | |
| | | Significant | 0.0484 | |
| | | probability | | |
| | | (p-value), | | |
| | | two-sample t-test | | |
| | Week 4 | Number of subjects | 307 | 304 |
| | Visit 5 | Mean (standard | −0.38 | −0.23 |
| | (29 ± 2 days) | deviation) | (0.785) | (0.737) |
| | | LS mean difference | −0.11 | |
| | | p-value, covariance | 0.0321 | |
| | | (ANCOVA) | | |
| | | p-value, two-sample | 0.0195 | |
| | | t-test | | |
| | Week 8 | Number of subjects | 288 | 294 |
| | Visit 6 | Mean (standard | −0.32 | −0.18 |
| | (57 + 3 days) | deviation) | (0.834) | (0.758) |
| | | LS mean difference | −0.10 | |
| | | | (0.055) | |
| | | p-value, covariance | 0.0711 | |
| | | (ANCOVA) | | |
| | | p-value, two-sample | 0.0239 | |
| | | t-test | | |
| TCSS | Week 8 | Number of subjects | 288 | 294 |
| | Visit 6 | Mean (standard | −0.82 | −0.56 |
| | (57 + 3 days) | deviation) | (1.664) | (1.645) |

| | |
|---|---|
| LS mean difference | −0.24 |
| p-value, covariance | 0.0452 |
| (ANCOVA) | |
| p-value, two-sample | 0.0638 |
| t-test | |

ANCOVA and p-value were calculated by adopting treatment, baseline score and clinical trial site as covariate variables.

3. Result of ODS and EDS Evaluation

After completion of investigational product administration for 8 weeks, mean changes from baselines in ODS and EDS as the subjective symptoms were investigated.

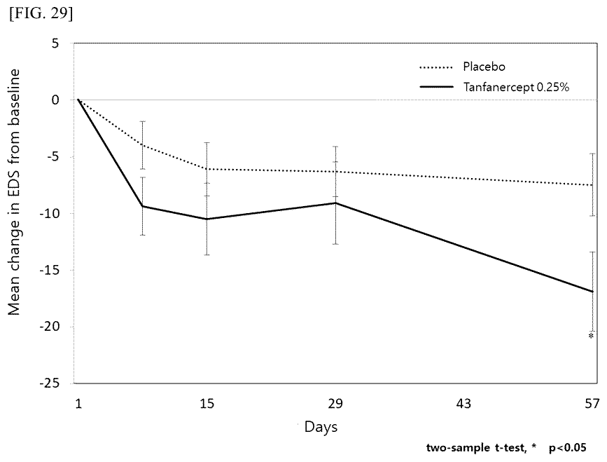

Table 25 shows mean change in EDS from baseline to week 8 with regard to subjects with/without experience of using artificial tears within 1 month from Visit 1 as well as all subjects. Further, FIG. 29 illustrates mean change from the baseline in EDS of the subjects having experience of using artificial tears within 1 month from Visit 1 at week 8, among the above subjects.

As shown in Table 25 and FIG. 29, with regard to randomly assigned subjects (ITT Population, observed data only), the test drug did not exhibit significant improvement on EDS, a subjective indicator of dry eye syndrome, relative to placebo. However, it was confirmed that a patient group relatively sensitive to symptoms, who needed to use artificial tears within 30 days from Visit 1 (−14 day), exhibited significant effects (p=0.0334).

All subjects exhibited improvement on ODS at week 2 and week 4.

In a questionnaire on subjective symptoms of the patient, a significant difference in ODS, which was a primary endpoint, was not confirmed in ITT population. However, when patients who are relatively sensitive to symptoms with experience using artificial tears within 1 month from Visit 1 were subjected to sub-group analysis, a significant difference in EDS could be confirmed.

TABLE 25
Mean change in EDS from baseline to Week 8

| | EDS change from baseline at week 8 | | | | | | |
|---|---|---|---|---|---|---|---|
| | | | Mean | | | | |
| | N | | (standard deviation) | | LS mean | p-value | |
| Group | Placebo | Test drug | Placebo | Test drug | difference | ANCOVA | t-test |
| ITT Population | 294 | 293 | −11.3 ± 23.01 | −12.9 ± 24.74 | −1.0 | 0.5771 | 0.4130 |
| No use of | 227 | 238 | −12.4 ± 23.11 | −12.0 ± 24.42 | +1.0 | 0.6484 | 0.8454 |
| artificial tears | | | | | | | |
| within 1 month | | | | | | | |
| from Visit 1 | | | | | | | |
| Use of artificial | 67 | 55 | −7.50 ± 22.42 | −16.9 ± 25.92 | −7.1 | 0.1038 | 0.0334* |
| tears within 1 | | | | | | | |
| month from | | | | | | | |
| Visit 1 | | | | | | | |

Two-sample t-test * p < 0.05

4. Clinical Trial Result of Sub-Group Patient Group

The patients participating in the clinical trial were divided into sub-groups on the basis of baseline severity, that is, severity of disease at the start of the clinical trial, followed by analysis of sign and symptom relevant indicators.

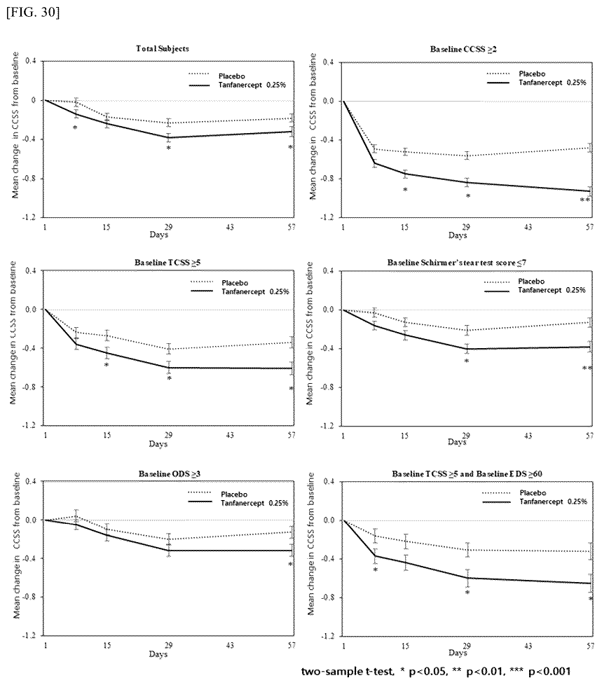

Table 26 and FIG. 30 are a summary of mean changes from baseline in CCSS at week 8 with respect to disease severity.

TABLE 26
Mean change in CCSS from baseline to Week 8 stratified by baseline disease severity

| | CSS change from baseline at week 8 | | | | | | |
|---|---|---|---|---|---|---|---|
| | | | Mean | | | | |
| | Number | | (standard deviation) | | LS mean | p-value | |
| Group | Placebo | Test drug | Placebo | Test drug | difference | ANCOVA | t-test |
| ITT | 294 | 288 | −0.18 ± 0.758 | −0.32 ± 0.834 | −0.10 | 0.0711 | 0.0239* |
| Population | | | | | | | |
| Baseline | 263 | 252 | −0.25 ± 0.723 | −0.42 ± 0.817 | −0.01 | 0.0803 | 0.0138* |
| CCSS ≥ 1 | | | | | | | |
| Baseline | 87 | 96 | −0.48 ± 0.768 | −0.93 ± 0.797 | −0.40 | 0.0004 | 0.0001*** |
| CCSS ≥ 2 | | | | | | | |
| Baseline | 271 | 262 | −0.25 ± 0.706 | −0.40 ± 0.819 | −0.09 | 0.1216 | 0.0258* |
| TCSS ≥ 4 | | | | | | | |
| Baseline | 163 | 150 | −0.33 ± 0.762 | −0.61 ± 0.835 | −0.19 | 0.0199 | 0.0031** |
| TCSS ≥ 5 | | | | | | | |
| Baseline | 229 | 220 | −0.13 ± 0.731 | −0.38 ± 0.825 | −0.19 | 0.0016 | 0.0008*** |
| Schirmer's | | | | | | | |
| score after | | | | | | | |
| CAE ® ≤ 7 | | | | | | | |
| Baseline | 110 | 96 | −0.12 ± 0.755 | −0.43 ± 0.797 | −0.27 | 0.0024 | 0.0047** |
| Schirmer's | | | | | | | |
| score after | | | | | | | |
| CAE ® ≤ 3 | | | | | | | |
| Baseline | 163 | 162 | −0.13 ± 0.811 | −0.32 ± 0.791 | −0.17 | 0.0247 | 0.0264* |
| ODS ≥ 3 | | | | | | | |
| Baseline | 95 | 79 | −0.32 ± 0.792 | −0.67 ± 0.784 | −0.27 | 0.0142 | 0.0035** |
| TCSS ≥ 5 | | | | | | | |
| and ODS ≥ 3 | | | | | | | |
| Baseline | 84 | 90 | −0.32 ± 0.779 | −0.65 ± 0.888 | −0.25 | 0.0301 | 0.0092** |
| TCSS ≥ 5 | | | | | | | |
| and EDS ≥ 60 | | | | | | | |

Two-sample t-test *p < 0.05, **p < 0.01, ***p < 0.001

As shown in Table 26 and FIG. 30, it was confirmed that efficacy of the test drug was more obviously demonstrated than the placebo in the patient group with higher baseline disease severity.

Analyzed results of the total subjects participating in the clinical trial demonstrated that a difference between the test drug and the placebo in terms of CCSS began to appear from 1 week after dosage and was continued until 8 weeks (p=0.0239, two-sample t-test).

Among the total patients, only the patients with severe corneal damage who have baseline TCSS of 5 or more were analyzed. From the analyzed results, significance relative to placebo (p=0.0031) was confirmed in terms of CCSS improvement.

Further, only the patients having damage of central corneal portion at the start of dosage, that is, baseline CCSS of 2 points or more were analyzed. From the analyzed results, significance was confirmed (p=0.0001).

Further, only the patients having the baseline Schirmer's tear test score in the range of 1 to 7, that is, relatively less tear secretion were analyzed. From the analyzed results, significance was also confirmed (p=0.0008).

Consequently, with regard to signs, it could be understood that efficacy of the test drug is more obviously demonstrated than the placebo in the patient group having high baseline severity. Likewise, similar results were also observed in the patient group with high symptom baseline severity or the patient group with high severity of a combination of sign and symptom baseline scores.

Further, as a result of analysis of the total patients, the test drug showed significant improvement on corneal damage at week 8, relative to the placebo, in terms of total corneal staining score (TCSS), a sum of CCSS, ICSS and SCSS of three corneal regions (p=0.0452, ANCOVA). It could be seen that therapeutic effects of the test drug in terms of TCSS are more obviously demonstrated in analysis of the patient group having high baseline disease severity among the total patients.

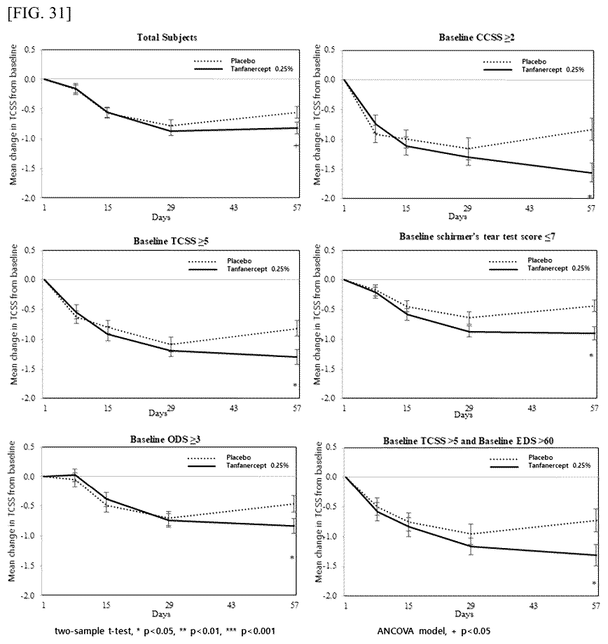

The following Table 27 and FIG. 31 illustrate mean change in TCSS stratified by baseline disease severity from the baseline to week 8.

Among the total subjects, only the patients having baseline TCSS of 5 or more, that is, severe corneal damage were analyzed. From the analyzed results, significance relative to placebo was confirmed in terms of TCSS improvement 8 weeks after dosing (p=0.0109).

Further, only the patients having damage of the central corneal region at the start of dosing, that is, baseline CCSS of 2 or more were analyzed. From the analyzed results, significance was confirmed (p=0.0019).

Further, only the patients having baseline STT in the range of 1 to 7, that is, relatively less tear secretion, were analyzed. From the analyzed results, significance was also confirmed (p=0.0025).

In addition, likewise, similar results were also observed in the patient group with high symptom baseline severity or the patient group with high severity of a combination of sign and symptom baselines. These results were also confirmed to be substantially the same in two-sample t-test statistical analysis results.

TABLE 27
Mean change in TCSS from baseline to week 8 stratified by baseline disease severity

| | TCSS change from baseline at week 8 | | | | | | |
|---|---|---|---|---|---|---|---|
| | | | Mean | | | | |
| | Number | | (standard deviation) | | LS mean | p-value | |
| Group | Placebo | Test drug | Placebo | Test drug | difference | ANCOVA | t-test |
| ITT | 294 | 288 | −0.56 ± 1.645 | −0.82 ± 1.664 | −0.24 | 0.0452 | 0.0638 |
| Population | | | | | | | |
| Baseline | 263 | 252 | −0.70 ± 1.599 | −0.95 ± 1.627 | −0.24 | 0.0552 | 0.0738 |
| CCSS ≥ 1 | | | | | | | |
| Baseline | 87 | 96 | −0.83 ± 1.703 | −1.56 ± 1.518 | −0.73 | 0.0019 | 0.0025** |
| CCSS ≥ 2 | | | | | | | |
| Baseline | 271 | 262 | −0.70 ± 1.571 | −0.96 ± 1.610 | −0.22 | 0.0695 | 0.0610 |
| TCSS ≥ 4 | | | | | | | |
| Baseline | 163 | 150 | −0.82 ± 1.661 | −1.30 ± 1.586 | −0.43 | 0.0109 | 0.0089** |
| TCSS > 5 | | | | | | | |
| Baseline | 229 | 220 | −0.44 ± 1.568 | −0.90 ± 1.623 | −0.40 | 0.0025 | 0.0028** |
| Schirmer's | | | | | | | |
| score after | | | | | | | |
| CAE ® ≤ 7 | | | | | | | |
| Baseline | 110 | 96 | −0.53 ± 1.662 | −0.92 ± 1.566 | −0.42 | 0.0343 | 0.0863 |
| Schirmer's | | | | | | | |
| score after | | | | | | | |
| CAE ® ≤ 3 | | | | | | | |
| Baseline | 163 | 162 | −0.47 ± 1.767 | −0.84 ± 1.607 | −0.40 | 0.0161 | 0.0473* |
| ODS ≥ 3 | | | | | | | |
| Baseline | 95 | 79 | −0.77 ± 1.722 | −1.42 ± 1.471 | −0.60 | 0.0109 | 0.0095** |
| TCSS ≥ 5 | | | | | | | |
| and ODS ≥ 3 | | | | | | | |
| Baseline | 84 | 90 | −0.73 ± 1.673 | −1.31 ± 1.636 | −0.63 | 0.0075 | 0.0222* |
| TCSS > 5 | | | | | | | |
| and EDS ≥ 60 | | | | | | | |

Two-sample t-test *p < 0.05, **p < 0.01, ***p < 0.001

1.-17. (canceled)

18. A method for treatment of dry eye syndrome, comprising administering an ophthalmic composition, which comprises tanfanercept as an active ingredient, to a patient with moderate-to-severe dry eye.

19. The method according to claim 18, wherein the patient has a corneal staining score of 2 or more on at least one region of Inferior Corneal, Central Corneal and Superior Corneal and a Schirmer's tear test score of 1 to 7.

20. The method according to claim 18, wherein improvement of sign of the moderate-to-severe dry eye begins to appear within 8 weeks after administration of the ophthalmic composition.

21. The method according to claim 20, wherein the improvement of sign is determined on the basis of reduction in a total corneal staining score (TCSS) and a central corneal staining score (CCSS).

22. The method according to claim 18, wherein improvement of symptom of the moderate-to-severe dry eye begins to appear within 8 weeks after administration of the ophthalmic composition.

23. The method according to claim 22, wherein the improvement of symptom is determined on the basis of reduction of an eye dryness score (EDS).

24. The method according to claim 18, wherein the patient has CCSS of 2 or more before administration of the ophthalmic composition.

25. The method according to claim 18, wherein the patient has TCSS of 5 or more before administration of the ophthalmic composition.

26. The method according to claim 18, wherein the patient has a Schirmer's tear test (STT) score of 1 to 7 before administration of the ophthalmic composition.

27. The method according to claim 18, wherein the patient has STT score of 1 to 7 before administration of the ophthalmic composition.

28. The method according to claim 18, wherein the patient has an ocular discomfort score (ODS) of 3 or more before administration of the ophthalmic composition.

29. The method according to claim 18, wherein the patient has TCSS of 5 or more and ODS of 3 or more before administration of the ophthalmic composition.

30. The method according to claim 18, wherein the patient has TCSS of 4 or more and EDS of 40 or more before administration of the ophthalmic composition.

31. The method according to claim 18, wherein the ophthalmic composition is administered in the form of an eye drop once or more a day.

32. The method according to claim 18, wherein the ophthalmic composition is administered in the form of an eye drop twice a day.

33. The method according to claim 18, wherein the ophthalmic composition comprises tanfanercept and a buffer system at pH 5.0 to pH 6.5, and the ophthalmic composition is substantially free of a stabilizer.

34. The method according to claim 18, wherein the ophthalmic composition comprises 0.01 to 1% (w/v) of tanfanercept.
